(19) United States
(12) Patent Application Publication
(10) Pub. No.: US 2006/0128708 A1
(43) Pub. Date: Jun. 15, 2006

(54) Antagonizing an adenosine A2A receptor to ameliorate one or more components of addictive behavior

(75) Inventors: Ivan F. Diamond, Berkeley, CA (US); Adrienne S. Gordon, Kensington, CA (US)

Correspondence Address: QUINE INTELLECTUAL PROPERTY LAW GROUP, P.C., P O BOX 458, ALAMEDA, CA, 94501, US

(73) Assignee: The Regents of the University of California

(21) Appl. No.: 11/153,725

(22) Filed: Jun. 14, 2005

Related U.S. Application Data
(60) Provisional application No. 60/581,143, filed on Jun. 17, 2004.

(51) Int. Cl. A61K 31/53; A61K 31/519; A61K 31/522

(52) U.S. Cl. 514/243; 514/263.34; 514/259.31; 514/263.33

(57) ABSTRACT

This invention provides a method of mitigating/ameliorating one or more components of addictive behavior associated with chronic consumption of a substance of abuse, or withdrawal therefrom. The method typically involves administering to a subject in need thereof an adenosine A2A receptor antagonist in an amount sufficient to ameliorate said one or more components of addictive behavior.

### CROSS-REFERENCE TO RELATED APPLICATIONS

This application claims benefit of and priority to U.S. Ser. No. 60/581,143, filed on Jun. 17, 2004, which is incorporated herein by reference in its entirety for all purposes.

### STATEMENT AS TO RIGHTS TO INVENTIONS MADE UNDER FEDERALLY SPONSORED RESEARCH AND DEVELOPMENT

This work was supported in part by National Institutes of Health grants AA10030 and AA10039, by funds provided by the State of California for medical research on ethanol and substance abuse through the University of California and by a grant from the Department of the Army, DAMD 17-01-1-0803. The Government of the United States of America has certain rights in this invention.

### FIELD OF THE INVENTION

This invention pertains to the field of substance abuse. More particularly, this invention pertains to the discovery that adenosine A2A receptor antagonists can inhibit one or more components of addictive behavior associated with chronic consumption of a substance of abuse, or withdrawal therefrom

### BACKGROUND OF THE INVENTION

The abuse of ethanol and other “addictive substances” remains a major public health problem in the U.S. and throughout the world. Drug dependency is extremely difficult to escape. This is true whether the dependency is one based on ethanol, amphetamine, barbiturates, benzodiazepines, cocaine, nicotine, opioids, and phencyclidine or the like. There is thus a need for an agent for decreasing or overcoming such addiction and/or one or more behavioral components (e.g., craving) associated with such addiction.

### SUMMARY OF THE INVENTION

This invention pertains to the discovery that adenosine A2A receptor antagonists can inhibit one or more components of addictive behavior associated with chronic consumption of a substance of abuse, or withdrawal therefrom. The method typically involves administering to a subject in need thereof an adenosine A2A receptor antagonist in an amount sufficient to ameliorate said one or more components of addictive behavior. Typically, the A2A receptor antagonist is not a xantheine (e.g., caffeine or a caffeine derivative). In certain embodiments, the adenosine A2A receptor antagonist specifically inhibits A2A receptors and has a substantially reduced effect on other adenosine receptors (e.g. A1 receptors).

Thus in one embodiment, this invention provides a method of mitigating one or more components of addictive behavior associated with chronic consumption of a substance of abuse, with cessation of such chronic consumption, and/or withdrawal therefrom, by a mammal. The method typically involves administering to the mammal exhibiting one or more components of addictive behavior an adenosine A2A receptor antagonist in an amount sufficient to ameliorate the one or more components of addictive behavior. In certain embodiments the A2A receptor antagonist is not caffeine and/or not a caffeine derivative. In certain embodiments the adenosine A2A receptor antagonist includes, but is not limited to (−)-R,S)-mefloquine, 3,7-Dimethyl-1-propargylxanthine (DMPX), 3-(3-hydroxypropyl)-7-methyl-8-(m-methoxystyryl)-1-propargylxanthine (MX2), 3-(3-hydroxypropyl)-8-(3-methoxystyryl)-7-methyl-1-propargylxanthin phosphate disodium salt (MSX-3), 7-methyl-8-styrylxanthine derivatives, SCH 58261, KW-6002, aminofuryltriazolo-triazinylaminoethylphenol (ZM 241385), and 8-chlorostyrylcaffeine, KF17837, VR2006, istradefylline, VER-11135, VER-6409, VER 6440, VER 6489, VER 6623, VER 6947, VER 7130, VER 7146, VER 7448, VER 7835, VER 8177, pyrazolo [4,3-e]1,2,4-triazolo[1,5-c]pyrimidines, 5-amino-imidazolo-[4,3-e]-1,2,4-triazolo[1,5-c]pyrimidines, and the like. In certain embodiments the antagonist does not substantially antagonize the adenosine A1A receptor. In various embodiments the can be ethanol, an opiate, a cannabinoid, nicotine, a stimulant, and the like. In various embodiments the substance of abuse can be morphine, heroin, marijuana, hashish, cocaine, amphetamines, and the like. In certain embodiments the substance of abuse is ethanol. In various embodiments the component of addictive behavior is chronic self-administration of the substance of abuse and/or craving for the substance of abuse, and/or reinstatement of seeking behavior for the substance of abuse. In certain embodiments the mammal is a mammal engaging in chronic consumption of a substance of abuse. In certain embodiments mammal is a mammal that has ceased chronic consumption of a substance of abuse. In certain embodiments the mammal is a mammal undergoing one or more symptoms of withdrawal. In certain embodiments the mammal is a human, e.g., a human not suffering from Parkinson's disease. In various embodiments the antagonist is administered systemically (e.g., by a route such as oral administration, nasal administration, rectal administration, intraperitoneal injection, intravascular injection, subcutaneous injection, transcutaneous administration, inhalation administration, intramuscular injection, and the like). Typically, the antagonist is formulated as a unit dosage formulation, e.g. as a time-release formulation. In certain embodiments the method further comprises administering a dopamine D2 receptor antagonist or agonist in conjunction with the adenosine A2A receptor antagonist. The D2 receptor antagonist or agonist can be administered before, during, or after the A2A receptor antagonist. Suitable D2 receptor antagonists include, but are not limited to butaclamol, chlorpromazine, domperidone, fluphenazine, haloperidol, heteroaryl piperidines, metoclopramide, olanzapine, perospirone hydrochloride hydrate, phenothiazine, pimozide, quetiapine, risperidone, sertindole, sulpiride, ziprasidone, zotepine, and the like.

This invention also provides a composition for mitigating one or more components of addictive behavior associated with chronic consumption of a substance of abuse, cessation of such consumption, or withdrawal therefrom, by a mammal. The composition typically comprises an adenosine A2A receptor antagonist; and a dopamine D2 receptor antagonist. Suitable A2A receptor antagonists and/or D2 receptor antagonists include, but are not limited to those described above.

Also provided are kits for mitigating one or more components of addictive behavior associated with chronic consumption of a substance of abuse, cessation of such consumption, and/or withdrawal therefrom, by a mammal. The kits typically comprise a container containing one or more adenosine A2A receptor antagonists where at least one of the one or more adenosine A2A receptor antagonists is not caffeine and/or a caffeine derivative ; and instructional materials teaching the use of the adenosine A2A receptor antagonists in the treatment of substance abuse in a mammal. Suitable A2A receptor antagonists include, but are not limited to those described above. In various embodiments the the antagonist does not substantially antagonize the adenosine A1A receptor. In various embodiments the substance of abuse includes, but is not limited to ethanol, an opiate, a cannabinoid, nicotine, a stimulant, morphine, heroin, marijuana, hashish, cocaine, amphetamines, and the like. Various component of addictive behavior include, but are not limited to chronic self-administration of the substance of abuse, craving for the substance of abuse, reinstatement of seeking behavior for the substance of abuse, and the like. In certain embodiments the antagonist is formulated for administration by a route such as oral administration, nasal administration, rectal administration, intraperitoneal injection, intravascular injection, subcutaneous injection, transcutaneous administration, inhalation administration, intramuscular injection, and the like. In various embodiments the antagonist is formulated as a unit dosage formulation, e.g., as a time-release formulation.

Also provided are methods of screening for an agent that inhibits one or more components of addictive behavior associated with chronic consumption of a substance of abuse. The method typically involves providing one or more test agents; and screening the test agents for the ability to inhibit adenosine A2A receptor expression or activity where inhibition of adenosine A2A receptor expression or activity indicates that the one or more test agents are candidate agents for inhibiting one or more components of addictive behavior associated with chronic consumption of a substance of abuse, or withdrawal therefrom. In certain embodiments the screening comprises screening the test agent for the ability to bind to an A2A receptor, and, optionally further screening the test agent for the ability to inhibit operant self-administration of the substance of abuse. In certain embodiments the screening comprises further screening the test agent for the ability to inhibit reinstatement of seeking behavior for the substance of abuse. In certain embodiments the substance of abuse is selected from the group consisting of ethanol, an opiate, a cannabinoid, nicotine, and a stimulant. In certain embodiments the substance of abuse is selected from the group consisting of morphine, heroin, marijuana, hashish, cocaine, amphetamines, and the like.

This invention also provides a method of screening for an agent that inhibits one or more components of addictive behavior associated with chronic consumption of a substance of abuse where the method involves providing one or more putative adenosine A2A receptor antagonists; and screening the test agents for the ability to inhibit one or more components of an addictive behavior associated with chronic consumption of a substance of abuse or withdrawal therefrom. In certain embodiments the screening comprises screening the test agent for the ability to inhibit operant self-administration of the substance of abuse. In certain embodiments the screening comprises further screening the test agent for the ability to inhibit reinstatement of seeking behavior for the substance of abuse. The substance of abuse can include, but is not limited to any of the substances of abuse described herein.

In certain instances, in any of the embodiments described herein, it is contemplated that an adenosine A2A receptor agonist may be utilized instead of the A2A receptor antagonist.

### DEFINITIONS

The term “substance abuse” refers to the use of a substance, generally chemical in nature, in a manner which is generally considered improper in view of the intended use of the substance. Substance abuse is becoming extremely widespread in today's world. Indeed, many consider the problem of substance abuse to have reached epidemic proportions. As substance abuse becomes more widespread the catastrophic effects of such substance abuse become more and more apparent to members of society. As a result of an ever increasing awareness of the catastrophic effects of substance abuse, society begins to seek methods for preventing and treating such substance abuse.

The term “substance of abuse” typically refers to a substance that is psychoactive and that induces tolerance and/or addiction. Substances of abuse include, but are not limited to stimulants (e.g. cocaine, amphetamines), opiates (e.g. morphine, heroin), cannabinoids (e.g. marijuana, hashish), nicotine, alcohol, substances that mediate agonist activity at the dopamine D2 receptor, and the like. Substances of abuse include, but are not limited to addictive drugs. In the case of addictive over-consumption, food, sugar, and the like can be considered a substance of abuse.

A “dopamine receptor antagonist” refers to a substance that reduces or blocks activity mediated by a dopamine receptor in response to the cognate ligand of that receptor. Thus, for example, a dopamine receptor antagonist will reduce or eliminate the activity of dopamine mediated by a dopamine receptor and associated pathway(s). The activity of the antagonist can be directly at the receptor, e.g., by blocking the receptor or by altering receptor configuration or activity of the receptor. The activity of the antagonist can also be at other points (e.g. at one or more second messengers, kinases, etc.) in a metabolic pathway that mediates the receptor activity.

An “adenosine A2a receptor antagonist” refers to a substance that reduces or blocks activity mediated by an adenosine A2a receptor in response to the cognate ligand of that receptor. The activity of the antagonist can be directly at the receptor, e.g., by blocking the receptor or by altering receptor configuration or activity of the receptor. The activity of the antagonist can also be at other points (e.g. at one or more second messengers, kinases, etc.) in a metabolic pathway that mediates the receptor activity.

The phrase “in conjunction with” when used in reference to the use adenosine A2A receptor antagonists and dopamine D2 receptor antagonists indicates that the A2A antagonist and the D2 antagonist are administered so that there is at least some chronological overlap in their physiological activity on the organism. Thus the A2A antagonist and the D2 antagonist can be administered simultaneously and/or sequentially. In sequential administration there may even be some substantial delay (e.g., minutes or even hours or days) before administration of the second agent as long as the first administered agent has exerted some physiological alteration on the organism when the second administered agent is administered or becomes active in the organism.

The phrase “does not substantially antagonize a receptor”, e.g. when used with reference to the impact of a agent on a receptor (e.g. the adenosine A1A receptor) indicates that the agent does not reduce activity of the receptor, e.g. in response to cognate or other “agonistic” ligand by more than 50%, preferably activity is not reduced by more than 20%, more preferably activity is not reduced by more than 10%, and most preferably activity is not reduced by more than 5% or 1%.

### BRIEF DESCRIPTION OF THE DRAWINGS

FIGS. 1A and 1B show the bimodal effect of the A2A antagonist DMPX on EtOH self-administration. Results represent means±SEM of number of lever presses (FIG. 1A), and g/kg EtOH consumption (FIG. 1B) during the 30 min FR3 session of operant responding under the different DMPX doses tested. The A2A antagonist was administered ip 20 min prior to the session. *Significantly different as compared with saline treatment (ANOVA with LSD post-hoc comparisons, p<0.05, n=18 for saline group, n=10 for 1, 3, and 10 mg/kg, n=8 for 5, 7, and 20 mg/kg).

FIGS. 2A and 2B shows that the A1 antagonist DPCPX did not affect EtOH self-administration. Results represent means±SEM of number of lever presses (A) and g/kg EtOH consumption (B) during the 30 min FR3 session of operant responding under the different DPCPX doses tested. The A1 antagonist was administered ip 15 min prior to the session (n=7/group).

FIGS. 3A and 3B show that the D2 antagonist eticlopride dose-dependently decreased EtOH self-administration. Results represent means±SEM of number of lever presses (FIG. 3A) and g/kg EtOH consumption (FIG. 3B) during the 30 min FR3 session of operant responding under the different eticlopride doses tested. The D2 antagonist was administered sc 25 min prior to the session. *, ** Significantly different as compared with saline treatment (ANOVA with LSD post-hoc comparisons, p<0.05 and p<0.01, respectively; n=5/group).

### DETAILED DESCRIPTION

This invention pertains to the discovery that antagonists of the adenosine A2A receptor can inhibit (reduce or block) one or more components of behavior associated with addiction to a substance (e.g., to a substance of abuse). It was a surprising discovery inhibition of the A2A receptor, e.g., by systemic administration of an adenosine A2A antagonist blocks operant self-administration of a substance of abuse (e.g., ethanol). In addition, it was demonstrated that adenosine mediates reinstatement of seeking behavior (operant self-administration) and that this is also blocked by an A2A antagonist administered systemically. Reinstatement is considered to be a more direct measure of craving or addiction for alcohol, or other substances of abuse. In addition, it was demonstrated that an A2A antagonist administered directly into the nucleus accumbens in the brain of rats addicted to heroin prevents reinstatement of heroin self-administration by self-injection into veins. The nucleus accumbens is the brain region presumed to mediate craving for addicting drugs. In contrast, adenosine A1 receptor antagonists appear ineffective in this context.

Thus, this invention provides methods of mitigating one or more components of addictive behavior associated with chronic consumption of a substance of abuse, or withdrawal therefrom, by a mammal (e.g. a human) where the method involves administering to the mammal one or more adenosine A2A receptor antagonists in an amount sufficient to ameliorate one or more components of addictive behavior (e.g. craving, seeking behavior, anxiety, chronic self-administration, etc.). Typically the A2A receptor antagonist is not caffeine and, in certain embodiments, the A2A receptor antagonist is not a xanthine or a modified or derivatized xanthine.

Without being bound to a particular theory, it is believed that A2A receptor antagonists can be effective in the treatment of addictive behaviors (addiction) to any of a wide variety of addictive materials. Such materials, include, but are not limited to stimulants (e.g. cocaine, amphetamines), opiates (e.g. morphine, heroin), cannabinoids (e.g. marijuana, hashish), nicotine, alcohol, substances that mediate agonist activity at the dopamine D2 receptor, and the like. In certain instances, food and/or sugar can be regarded as a substance of abuse (e.g. in compulsive eating disorders).

Typically one or more adenosine A2A receptor antagonists will be administered to a mammal, more typically to a human to ameliorate one or more behaviors associated with addiction, e.g., to a substance of abuse or withdrawal from such a substance. Most typically, the adenosine A2A receptor antagonists will be administered to reduce self administration and/or seeking behavior and/or to reduce cravings and/or anxiety. In certain embodiments, the subjects will be subjects that are not being treated for Parkinsons syndrome or other neurological disorders (other than those associated with addictive behavior).

I. Adenosine A2A Receptor Antagonists.

A number of adenosine A2A receptor antagonists are known to those of skill in the art and can be used individually or in conjunction in the methods described herein. Such antagonists include, but are not limited to (−)-R,S)-mefloquine (the active enantiomer of the racemic mixture marketed as Mefloquine™), 3,7-Dimethyl-1-propargylxanthine (DMPX), 3-(3-hydroxypropyl)-7-methyl-8-(m-methoxystyryl)-1-propargylxanthine (MX2), 3-(3-hydroxypropyl)-8-(3-methoxystyryl)-7-methyl-1-propargylxanthin phosphate disodium salt (MSX-3, a phosphate prodrug of MSX-2), 7-methyl-8-styrylxanthine derivatives, SCH 58261, KW-6002, aminofuryltriazolo-triazinylaminoethylphenol (ZM 241385), and 8-chlorostyrylcaffeine, KF17837, VR2006, istradefylline, the VERNALIS drugs such as VER 6489, VER 6623, VER 6947, VER 7130, VER 7146, VER 7448, VER 7835, VER 8177VER-11135, VER-6409, VER 6440, VER 6489, VER 6623, VER 6947, VER 7130, VER 7146, VER 7448, VER 7835, VER 8177, pyrazolo [4,3-e]1,2,4-triazolo[1,5-c]pyrimidines, and 5-amino-imidazolo-[4,3-e]-1,2,4-triazolo[1,5-c]pyrimidines., and the like. These adenosine A2A receptor antagonists are intended to be illustrative and not limiting.

In certain embodiments, adenosine A2A receptor antagonists are antagonists that have substantially less effect on the adenosine A1 receptor(s). In certain embodiments, the antagonists show at least 2 fold, preferably at least 5 fold, and more preferably at least 10 fold greater inhibitory activity on the A2A receptor as compared to the adenosine A1 receptor.

II. Use With Dopamine D2 Receptor Antagonists.

In certain embodiments, this invention contemplate the use of adenosine A2A receptor antagonists in conjunction with one or more dopamine D2 receptor antagonists.

Dopamine receptor antagonists are well known to those of skill in the art and include, but are not limited to butaclamol, chlorpromazine, domperidone, fluphenazine, haloperidol, heteroaryl piperidines, metoclopramide, olanzapine, perospirone hydrochloride hydrate, phenothiazine, pimozide, quetiapine, risperidone, sertindole, sulpiride, ziprasidone, zotepine, and the like.

In certain embodiments the dopamine D2 receptor antagonists and the adenosine A2A receptor antagonists are formulated as a single “compound” formulation. This can be accomplished by any of a number of known methods. For example, the A2A receptor antagonists and the D2 receptor antagonists can be combined in a single pharmaceutically acceptable excipient. In another approach the A2A receptor antagonists and the D2 receptor antagonists can be formulated in separate excipients that are microencapsulated and then combined, or that form separate laminae in a single pill, and so forth.

In certain embodiments the dopamine D2 receptor antagonists and the adenosine A2A receptor antagonists are joined directly together or are joined together by a “tether” or “linker” to form a single compound. Without being bound to a particular theory, it is believed that such joined antagonists provide improved specificity/selectivity.

A number of chemistries for linking molecules directly or through a linker/tether are well known to those of skill in the art. The specific chemistry employed for attaching the D2 receptor antagonist(s) and the A2A receptor antagonists to form a bifunctional antagonist depends on the chemical nature of the antagnoists(s) and the “interligand” (inter-antagonist) spacing desired. Various D2 receptor and/or A2A receptor antagonists typically contain a variety of functional groups (e.g. carboxylic acid (COOH), free amine (—NH2), and the like), that are available for reaction with a suitable functional group on a linker or on the other antagonist to bind the antagonists together.

Alternatively, the antagonist(s) can be derivatized to expose or attach additional reactive functional groups. The derivatization may involve attachment of any of a number of linker molecules such as those available from Pierce Chemical Company, Rockford Ill.

A “linker” or “tether”, as used herein, is a molecule that is used to join two or more ligands (e.g., receptor antagonists) to form a bi-functional or poly-functional antagonist. The linker is typically chosen to be capable of forming covalent bonds to all of the antagonist comprising the bi-functional or polyfunctional moiety. Suitable linkers are well known to those of skill in the art and include, but are not limited to, straight or branched-chain carbon linkers, heterocyclic carbon linkers, amino acids, nucleic acids, dendrimers, synthetic polymers, peptide linkers, peptide and nucleic acid analogs, carbohydrates, polyethylene glycol and the like. Where one or more of the antagonists are polypeptides, the linker can be joined to the constituent amino acids through their side groups (e.g., through a disulfide linkage to cysteine) or through the alpha carbon amino or carboxyl groups of the terminal amino acids.

In certain embodiments, a bifunctional linker having one functional group reactive with a group on the first D2 receptor antagonist and another group reactive with a functional group on the A2A receptor antagonist can be used to form a bifunctional antagonist. Alternatively, derivatization may involve chemical treatment of the antagonist(s), e.g., glycol cleavage of the sugar moiety of a glycoprotein, a carbohydrate, a or nucleic acid, etc., with periodate to generate free aldehyde groups. The free aldehyde groups can be reacted with free amine or hydrazine groups on a linker to bind the linker to the antagonist (see, e.g., U.S. Pat. No. 4,671,958). Procedures for generation of free sulfhydryl groups on polypeptide, such as antibodies or antibody fragments, are also known (See U.S. Pat. No. 4,659,839).

Where both the D2 receptor antagonist and the A2A receptor antagonist are both peptides, a bifunctional antagonist can be chemically synthesized or recombantly expressed as a fusion protein comprising both antagonists attached directly to each other or attached through a peptide linker.

In certain embodiments, lysine, glutamic acid, and polyethylene glycol (PEG) based linkers different length are used to couple the antagonists. Chemistry of the conjugation of molecules to PEG is well known to those of skill in the art (see, e.g., Veronese (20010 Biomaterials, 22: 405-417; Zalipsky and Menon-Rudolph (1997) Pp. 318-341 In: Poly(ethyleneglycol) Chemistry and Biological Applications. J. M. Harris and X. Zalipsky (eds)., Am. Chem. Soc. Washington, D.C.; Delgado et al. (1992) Drug Carrier Syst., 9: 249-304; Pedley et al. (1994) Br. J. Cancer, 70: 1126-113-0; Eyre and Farver (1991) Pp. 377-390 In: Textbook of Clinical Oncology, Holleb et al. (eds), Am. Cancer Soc., Atlanta Ga.; Lee et al. (1999) Bioconjug. Chem., 10: 973-981; Nucci et al. (1991) Adv. Drug Deliv., 6: 133-151; Francis et al. (1996) J. Drug Targeting, 3: 321-340).

In certain embodiments conjugation of the dopamine D2 receptor antagonists and the adenosine A2A receptor antagonists can be achieved by the use of such linking reagents such as glutaraldehyde, EDCI, terephthaloyl chloride, cyanogen bromide, and the like, or by reductive amination. In certain embodiments antagonists can linked via a hydroxy acid linker of the kind disclosed in WO-A-9317713. In certain embodiments PEG linkers can be utilized (see, e.g., Lee et al. (1999) Organic Lett., 1: 179-181, for the preparation of various PEG tethered drugs).

III. Pharmaceutical Formulations.

As explained herein, one or more symptoms associated with the chronic consumption of a substance of abuse (e.g. ethanol, opiates, barbiturates, etc.) can be mitigated by administration of one or more adenosine A2A receptor antagonists alone, or in certain embodiments, with the administration of one or more dopamine (D2) receptor antagonists. Similarly, one or more symptoms associated with withdrawal from the chronic consumption of a substance of abuse (e.g. ethanol) can be mitigated by administration of one or more adenosine A2A receptor antagonists alone in certain embodiments, with the administration of one or more dopamine (D2) receptor antagonists.

The adenosine A2A receptor antagonists and/or A2A receptor/D2 receptor antagonist combinations can be formulated in a number of forms including, but not limited to the form of the free acid the form of a salt, as a hydrate, etc. All forms are within the scope of the invention. Basic salts may be formed and are simply a more convenient form for use; in practice, use of the salt form inherently amounts to use of the acid form. The bases which can be used to prepare the salts include preferably those which produce, when combined with the free acid, pharmaceutically acceptable salts, that is, salts whose anions are non-toxic to the animal organism in pharmaceutical doses of the salts, so that the beneficial properties inherent in the free acid are not vitiated by side effects ascribable to the cations. Although pharmaceutically acceptable salts of the acid compound are preferred, all salts are useful as sources of the free acid form even if the particular salt per se is desired only as an intermediate product as, for example, when the salt is formed only for purposes of purification and identification, or when it is used as an intermediate in preparing a pharmaceutically acceptable salt by ion exchange procedures.

Such substances can be administered to a mammalian host in a variety of forms, i.e., they may be combined with various pharmaceutically acceptable inert carriers in the form of tablets, capsules, lozenges, troches, hard candies, powders, sprays, elixirs, syrups, injectable or eye drop solutions, and the like depending on the chosen route of administration, e.g., orally or parenterally. Parenteral administration in this respect includes administration by the following routes: intravenous, intramuscular, subcutaneous, intraocular, intrasynovial, transepithelial (including transdermal, ophthalmic, sublingual and buccal), topical (including ophthalmic, dermal, ocular, rectal, nasal inhalation via insufflation and aerosol), and rectal systemic. Oral administration is preferred.

Active compounds (e.g. adenosine A2A receptor antagonists) can be orally administered, for example, with an inert diluent or with an assimilable edible carrier, or they may be enclosed in hard or soft shell gelatin capsules, or they can be compressed into tablets, or they can be incorporated directly with the food of the diet. For oral therapeutic administration, the active compound can be incorporated with excipient and used in the form of ingestible tablets, buccal tablets, troches, capsules, elixirs, suspensions, syrups, wafers, and the like. Such compositions and preparations typically contain at least 0.1% of active compound. The percentage of the compositions and preparations may, of course, be varied and may conveniently be between about 2 to about 25% of the weight of the unit. The amount of active compound in such therapeutically useful compositions is such that a suitable dosage will be obtained. Preferred compositions or preparations according to the present invention are prepared so that an oral dosage unit form contains between about 1 and 1000 mg of active compound.

In certain embodiments, the tablets, troches, pills, capsules and the like can also contain the following: a binder such as polyvinylpyrrolidone, gum tragacanth, acacia, sucrose, corn starch or gelatin; an excipient such as calcium phosphate, sodium citrate and calcium carbonate; a disintegrating agent such as corn starch, potato starch, tapioca starch, certain complex silicates, alginic acid and the like; a lubricant such as sodium lauryl sulfate, talc and magnesium stearate; a sweetening agent such as sucrose, lactose or saccharin; or a flavoring agent such as peppermint, oil of wintergreen or cherry flavoring. Solid compositions of a similar type are also employed as fillers in soft and hard-filled gelatin capsules; preferred materials in this connection also include lactose or milk sugar as well as high molecular weight polyethylene glycols. When the dosage unit form is a capsule, it may contain, in addition to materials of the type described above, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye, flavoring such as cherry or orange flavor, emulsifying agents and/or suspending agents, as well as such diluents as water, ethanol, propylene glycol, glycerin and various like combinations thereof. Of course, any material used in preparing any dosage unit form should be pharmaceutically pure and substantially non-toxic in the amounts employed. In addition, the active compound may be incorporated into sustained-release preparations and formulations.

The active compound may also be administered parenterally or intraperitoneally. For purposes of parenteral administration, solutions in sesame or peanut oil or in aqueous propylene glycol can be employed, as well as sterile aqueous solutions of the corresponding water-soluble, alkali metal or alkaline-earth metal salts previously enumerated. Such aqueous solutions should be suitable buffered, if necessary, and the liquid diluent first rendered isotonic with sufficient saline or glucose. Solutions of the active compound as a free base or a pharmacologically acceptable salt can be prepared in water suitably mixed with a surfactant such as hydroxypropylcellulose. A dispersion can also be prepared in glycerol, liquid polyethylene glycols and mixtures thereof and in oils. Under ordinary conditions of storage and use, these preparations contain a preservative to prevent the growth of microorganisms. These particular aqueous solutions are especially suitable for intravenous, intramuscular, subcutaneous and intraperitoneal injection purposes. In this connection, the sterile aqueous media employed are all readily obtainable by standard techniques well-known to those skilled in the art.

The pharmaceutical forms suitable for injectable use include sterile aqueous solutions or dispersions and sterile powders for the extemporaneous preparation of sterile injectable solutions or dispersions. In all cases the form is desirably sterile and be fluid to the extent that easy syringability exists. It is desirably be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating the active compound in the required amount in the appropriate solvent with various of the other ingredients enumerated above, as required, followed by filtered sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique which yield a powder of the active ingredient plus any additional desired ingredient from the previously sterile-filtered solution thereof.

For purposes of topical administration, dilute sterile, aqueous solutions (usually in about 0.1% to 5% concentration), otherwise similar to the above parenteral solutions, are prepared in containers suitable for drop-wise administration to the eye. The therapeutic compounds of this invention may be administered to a mammal alone or in combination with pharmaceutically acceptable carriers. As noted above, the relative proportions of active ingredient and carrier are determined by the solubility and chemical nature of the compound, chosen route of administration and standard pharmaceutical practice. The dosage of the present therapeutic agents which will be most suitable for prophylaxis or treatment will vary with the form of administration, the particular compound chosen and the physiological characteristics of the particular patient under treatment. Generally, small dosages will be used initially and, if necessary, will be increased by small increments until the optimum effect under the circumstances is reached. Oral administration requires higher dosages. The compounds are administered either orally or parenterally, or topically as eye drops. Dosages can be readily determined by physicians using methods known in the art, using dosages typically determined from animal studies as starting points.

In certain embodiments, the dopamine receptor antagonist and/or the dopamine receptor antagonist are administered at a standard therapeutic dosage, more preferably at a substandard therapeutic dosage, still more preferably at about a threshold dosage, and most preferably at a sub threshold dosage, where the threshold dosage or subthreshold dosage is the threshold or subthreshold dosage for the respective antagonist administered alone. In certain particularly preferred embodiments, the adenosine A2A receptor antagonists are administered at a dosage lower than that dosage known to produce one or more adverse side-effects.

IV Kits.

This invention also contemplates kits for practice of the methods of this invention. Such kits typically include a container containing one or more adenosine A2A receptor antagonists as described herein. The kits typically additionally include instructional materials teaching the use of such antagonists to inhibit one or more components of addictive behavior associated with consumption of a substance of abuse. The instructional materials can teach preferred dosages, modes of administration, conterindications, and the like.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

V. Screening and/or Prescreening for Agents that Mitigate One or More Behavioral Components of Addiction to a Substance of Abuse or Withdrawal Therefrom.

As indicated above, in one aspect, this invention pertains to the discovery that adenosine receptor A2A antagonists can inhibit one or more components of addictive behavior associated with chronic consumption of a substance of abuse or withdrawal therefrom. Thus identification of putative adenosine A2A receptor inhibitors in effect identifies candidate agents for inhibiting one or more components of addictive behavior associated with chronic consumption of a substance of abuse, or withdrawal therefrom.

In certain embodiments, the screening methods involve screening test agents (e.g., putative adenosine A2A receptor antagonists) for the ability to inhibit expression and/or activity of an A2A receptor and/or to inhibit one or more components of addictive behavior (e.g. self-administration, craving, seeking, etc.)

Thus, in certain embodiments, the screening methods of this invention can involve contacting a mammalian test cell with a test agent; and detecting the expression or activity of an adenosine A2A receptor or other component of an A2A receptor signaling pathway (e.g., a beta/gamma dimer) where a difference A2A receptor expression or activity in the test cell, e.g. as compared to a control indicates that the test agent is a candidate for inhibiting gone or more components of addictive behavior.

Expression levels of a gene can be altered by changes in the transcription of the gene product (i.e. transcription of mRNA), and/or by changes in translation of the gene product (i.e. translation of the protein), and/or by post-translational modification(s) (e.g. protein folding, glycosylation, etc.). Thus preferred assays of this invention include assaying for level of transcribed mRNA (or other nucleic acids derived from nucleic acids that encode an A2A receptor or other component of an A2A receptor signaling pathway), level of translated protein, activity of translated protein, etc. Examples of such approaches are described below. These examples are intended to be illustrative and not limiting.

A) Nucleic-Acid Based Assays.

1) Target Molecules.

Changes in expression levels of an A2A receptor or other component of an A2A receptor signaling pathway can be detected by measuring changes in mRNA and/or a nucleic acid derived from the mRNA (e.g. reverse-transcribed cDNA, etc.) that encodes the A2A receptor or pathway component. In order to measure the expression level it is desirable to provide a nucleic acid sample for such analysis. In preferred embodiments the nucleic acid is found in or derived from a biological sample. The term “biological sample”, as used herein, refers to a sample obtained from an organism or from components (e.g., cells) of an organism or a cell or tissue culture.

The nucleic acid (e.g., mRNA nucleic acid derived from mRNA) is, in certain preferred embodiments, isolated from the sample according to any of a number of methods well known to those of skill in the art. Methods of isolating mRNA are well known to those of skill in the art. For example, methods of isolation and purification of nucleic acids are described in detail in by Tijssen ed., (1993) Chapter 3 of Laboratory Techniques in Biochemistry and Molecular Biology: Hybridization With Nucleic Acid Probes, Part I. Theory and Nucleic Acid Preparation, Elsevier, N.Y. and Tijssen ed.

In a preferred embodiment, the “total” nucleic acid is isolated from a given sample using, for example, an acid guanidinium-phenol-chloroform extraction method and polyA+mRNA is isolated by oligo dT column chromatography or by using (dT)n magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)).

Frequently, it is desirable to amplify the nucleic acid sample prior to assaying for expression level. Methods of amplifying nucleic acids are well known to those of skill in the art and include, but are not limited to polymerase chain reaction (PCR, see, e.g, Innis, et al., (1990) PCR Protocols. A guide to Methods and Application. Academic Press, Inc. San Diego,), ligase chain reaction (LCR) (see Wu and Wallace (1989) Genomics 4: 560, Landegren et al. (1988) Science 241: 1077, and Barringer et al. (1990) Gene 89: 117, transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874), dot PCR, and linker adapter PCR, etc.).

In a particularly preferred embodiment, where it is desired to quantify the transcription level (and thereby expression) of an A2A receptor or other component of an A2A receptor signaling pathway in a sample, the nucleic acid sample is one in which the concentration of the an A2A receptor or other component of an A2A receptor signaling pathway mRNA transcript(s), or the concentration of the nucleic acids derived from the an A2A receptor or other component of an A2A receptor signaling pathway mRNA transcript(s), is proportional to the transcription level (and therefore expression level) of that gene. Similarly, it is preferred that the hybridization signal intensity be proportional to the amount of hybridized nucleic acid. While it is preferred that the proportionality be relatively strict (e.g., a doubling in transcription rate results in a doubling in mRNA transcript in the sample nucleic acid pool and a doubling in hybridization signal), one of skill will appreciate that the proportionality can be more relaxed and even non-linear. Thus, for example, an assay where a 5 fold difference in concentration of the target mRNA results in a 3 to 6 fold difference in hybridization intensity is sufficient for most purposes.

Where more precise quantification is required appropriate controls can be run to correct for variations introduced in sample preparation and hybridization as described herein. In addition, serial dilutions of “standard” target nucleic acids (e.g., mRNAs) can be used to prepare calibration curves according to methods well known to those of skill in the art. Of course, where simple detection of the presence or absence of a transcript or large differences in nucleic acid concentration is desired, no elaborate control or calibration is required.

In the simplest embodiment, the sample comprising a nucleic acid encoding an A2A receptor or other component of an A2A receptor signaling pathway the total mRNA or a total cDNA isolated and/or otherwise derived from a biological sample. The nucleic acid may be isolated from the sample according to any of a number of methods well known to those of skill in the art as indicated above.

2) Hybridization-Based Assays.

Using the known nucleic acid sequences encoding an A2A receptor or other components of an A2A receptor signaling pathway, detecting and/or quantifying transcript(s) of these nucleic acids can be routinely accomplished using nucleic acid hybridization techniques (see, e.g., Sambrook et al. supra). For example, one method for evaluating the presence, absence, or quantity of reverse-transcribed cDNA involves a “Southern Blot”. In a Southern Blot, the DNA (e.g., reverse-transcribed an A2A receptor mRNA), typically fragmented and separated on an electrophoretic gel, is hybridized to a probe specific for that nucleic acid. Comparison of the intensity of the hybridization signal from the “test” probe with a “control” probe (e.g. a probe for a “housekeeping gene) provides an estimate of the relative expression level of the target nucleic acid.

Alternatively, the mRNA can be directly quantified in a Northern blot. In brief, the mRNA is isolated from a given cell sample using, for example, an acid guanidinium-phenol-chloroform extraction method. The mRNA is then electrophoresed to separate the mRNA species and the mRNA is transferred from the gel to a nitrocellulose membrane. As with the Southern blots, labeled probes are used to identify and/or quantify the target mRNA. Appropriate controls (e.g. probes to housekeeping genes) provide a reference for evaluating relative expression level.

An alternative means for determining the an A2A receptor or other component of an A2A receptor signaling pathway expression level is in situ hybridization. In situ hybridization assays are well known (e.g., Angerer (1987) Meth. Enzymol 152: 649). Generally, in situ hybridization comprises the following major steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application.

In some applications it is necessary to block the hybridization capacity of repetitive sequences. Thus, in some embodiments, tRNA, human genomic DNA, or Cot-1 DNA is used to block non-specific hybridization.

3) Amplification-Based Assays.

In another embodiment, amplification-based assays can be used to measure an A2A receptor or other component of an A2A receptor signaling pathway (transcription) level. In such amplification-based assays, the target nucleic acid sequences (i.e., a nucleic acid encoding an A2A receptor or other component of an A2A receptor signaling pathway) act as template(s) in amplification reaction(s) (e.g. Polymerase Chain Reaction (PCR) or reverse-transcription PCR (RT-PCR)). In a quantitative amplification, the amount of amplification product will be proportional to the amount of template (e.g., an A2A receptor-encoding mRNA) in the original sample. Comparison to appropriate (e.g. healthy tissue or cells unexposed to the test agent) controls provides a measure of the transcript level.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis et al. (1990) PCR Protocols, A Guide to Methods and Applications, Academic Press, Inc. N.Y.). One approach, for example, involves simultaneously co-amplifying a known quantity of a control sequence using the same primers as those used to amplify the target. This provides an internal standard that may be used to calibrate the PCR reaction.

One preferred internal standard is a synthetic AW106 cRNA. The AW106 cRNA is combined with RNA isolated from the sample according to standard techniques known to those of skill in the art. The RNA is then reverse transcribed using a reverse transcriptase to provide copy DNA. The cDNA sequences are then amplified (e.g., by PCR) using labeled primers. The amplification products are separated, typically by electrophoresis, and the amount of labeled nucleic acid (proportional to the amount of amplified product) is determined. The amount of mRNA in the sample is then calculated by comparison with the signal produced by the known AW106 RNA standard. Detailed protocols for quantitative PCR are provided in PCR Protocols, A Guide to Methods and Applications, Innis et al. (1990) Academic Press, Inc. N.Y.

4) Hybridization Formats and Optimization of Hybridization Conditions.

### a) Array-Based Hybridization Formats.

In one embodiment, the methods of this invention can be utilized in array-based hybridization formats. Arrays are a multiplicity of different “probe” or “target” nucleic acids (or other compounds) attached to one or more surfaces (e.g., solid, membrane, or gel). In a preferred embodiment, the multiplicity of nucleic acids (or other moieties) is attached to a single contiguous surface or to a multiplicity of surfaces juxtaposed to each other.

In an array format a large number of different hybridization reactions can be run essentially “in parallel.” This provides rapid, essentially simultaneous, evaluation of a number of hybridizations in a single “experiment”. Methods of performing hybridization reactions in array based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211).

Arrays, particularly nucleic acid arrays can be produced according to a wide variety of methods well known to those of skill in the art. For example, in a simple embodiment, “low density” arrays can simply be produced by spotting (e.g. by hand using a pipette) different nucleic acids at different locations on a solid support (e.g. a glass surface, a membrane, etc.).

This simple spotting, approach has been automated to produce high density spotted arrays (see, e.g., U.S. Pat. No. 5,807,522). This patent describes the use of an automated system that taps a microcapillary against a surface to deposit a small volume of a biological sample. The process is repeated to generate high-density arrays.

Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high density oligonucleotide arrays. Synthesis of high-density arrays is also described in U.S. Pat. Nos. 5,744,305, 5,800,992 and 5,445,934.

### b) Other Hybridization Formats.

As indicated above a variety of nucleic acid hybridization formats are known to those skilled in the art. For example, common formats include sandwich assays and competition or displacement assays. Such assay formats are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587.

Sandwich assays are commercially useful hybridization assays for detecting or isolating nucleic acid sequences. Such assays utilize a “capture” nucleic acid covalently immobilized to a solid support and a labeled “signal” nucleic acid in solution. The sample will provide the target nucleic acid. The “capture” nucleic acid and “signal” nucleic acid probe hybridize with the target nucleic acid to form a “sandwich” hybridization complex. To be most effective, the signal nucleic acid should not hybridize with the capture nucleic acid.

Typically, labeled signal nucleic acids are used to detect hybridization. Complementary nucleic acids or signal nucleic acids may be labeled by any one of several methods typically used to detect the presence of hybridized polynucleotides as described herein.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario) and Q Beta Replicase systems.

### c) Optimization of Hybridization Conditions.

Nucleic acid hybridization simply involves providing a denatured probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids, or in the addition of chemical agents, or the raising of the pH. Under low stringency conditions (e.g., low temperature and/or high salt and/or high target concentration) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

One of skill in the art will appreciate that hybridization conditions may be selected to provide any degree of stringency. In a preferred embodiment, hybridization is performed at low stringency to ensure hybridization and then subsequent washes are performed at higher stringency to eliminate mismatched hybrid duplexes. Successive washes may be performed at increasingly higher stringency (e.g., down to as low as 0.25×SSPE at 37° C. to 70° C.) until a desired level of hybridization specificity is obtained. Stringency can also be increased by addition of agents such as formamide. Hybridization specificity may be evaluated by comparison of hybridization to the test probes with hybridization to the various controls that can be present.

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular probes of interest.

In a preferred embodiment, background signal is reduced by the use of a blocking reagent (e.g., tRNA, sperm DNA, cot-1 DNA, etc.) during the hybridization to reduce non-specific binding. The use of blocking agents in hybridization is well known to those of skill in the art (see, e.g., Chapter 8 in P. Tijssen, supra.)

Methods of optimizing hybridization conditions are well known to those of skill in the art (see, e.g., Tijssen (1993) Laboratory Techniques in Biochemistry and Molecular Biology, Vol. 24: Hybridization With Nucleic Acid Probes, Elsevier, N.Y.).

Optimal conditions are also a function of the sensitivity of label (e.g., fluorescence) detection for different combinations of substrate type, fluorochrome, excitation and emission bands, spot size and the like. Low fluorescence background surfaces can be used (see, e.g., Chu (1992) Electrophoresis 13:105-114). The sensitivity for detection of spots (“target elements”) of various diameters on the candidate surfaces can be readily determined by, e.g., spotting a dilution series of fluorescently end labeled DNA fragments. These spots are then imaged using conventional fluorescence microscopy. The sensitivity, linearity, and dynamic range achievable from the various combinations of fluorochrome and solid surfaces (e.g., glass, fused silica, etc.) can thus be determined. Serial dilutions of pairs of fluorochrome in known relative proportions can also be analyzed. This determines the accuracy with which fluorescence ratio measurements reflect actual fluorochrome ratios over the dynamic range permitted by the detectors and fluorescence of the substrate upon which the probe has been fixed.

### d) Labeling and Detection of Nucleic Acids.

The probes used herein for detection of an A2A receptor or other component of an A2A receptor signaling pathway expression levels can be full length or less than the full length. Shorter probes are empirically tested for specificity. Preferred probes are sufficiently long so as to specifically hybridize with the target nucleic acid(s) under stringent conditions. The preferred size range is from about 20 bases to the length of the target mRNA, more preferably from about 30 bases to the length of the target mRNA, and most preferably from about 40 bases to the length of the target mRNA.

The probes are typically labeled, with a detectable label. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like, see, e.g., Molecular Probes, Eugene, Oreg., USA), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

A fluorescent label is preferred because it provides a very strong signal with low background. It is also optically detectable at high resolution and sensitivity through a quick scanning procedure. The nucleic acid samples can all be labeled with a single label, e.g., a single fluorescent label. Alternatively, in another embodiment, different nucleic acid samples can be simultaneously hybridized where each nucleic acid sample has a different label. For instance, one target could have a green fluorescent label and a second target could have a red fluorescent label. The scanning step will distinguish sites of binding of the red label from those binding the green fluorescent label. Each nucleic acid sample (target nucleic acid) can be analyzed independently from one another.

Suitable chromogens that can be employed include those molecules and compounds which absorb light in a distinctive range of wavelengths so that a color can be observed or, alternatively, which emit light when irradiated with radiation of a particular wave length or wave length range, e.g., fluorescers.

Detectable signal can also be provided by chemiluminescent and bioluminescent sources. Chemiluminescent sources include a compound which becomes electronically excited by a chemical reaction and can then emit light which serves as the detectable signal or donates energy to a fluorescent acceptor. Alternatively, luciferins can be used in conjunction with luciferase or lucigenins to provide bioluminescence.

Spin labels are provided by reporter molecules with an unpaired electron spin which can be detected by electron spin resonance (ESR) spectroscopy. Exemplary spin labels include organic free radicals, transitional metal complexes, particularly vanadium, copper, iron, and manganese, and the like. Exemplary spin labels include nitroxide free radicals.

The label can be added to the target (sample) nucleic acid(s) prior to, or after the hybridization. So called “direct labels” are detectable labels that are directly attached to or incorporated into the target (sample) nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization With Nucleic Acid Probes, P. Tijssen, ed. Elsevier, N.Y., (1993)).

Fluorescent labels are easily added during an in vitro transcription reaction. Thus, for example, fluorescein labeled UTP and CTP can be incorporated into the RNA produced in an in vitro transcription.

The labels can be attached directly or through a linker moiety. In general, the site of label or linker-label attachment is not limited to any specific position. For example, a label may be attached to a nucleoside, nucleotide, or analogue thereof at any position that does not interfere with detection or hybridization as desired. For example, certain Label-ON Reagents from Clontech (Palo Alto, Calif.) provide for labeling interspersed throughout the phosphate backbone of an oligonucleotide and for terminal labeling at the 3′ and 5′ ends. As shown for example herein, labels can be attached at positions on the ribose ring or the ribose can be modified and even eliminated as desired. The base moieties of useful labeling reagents can include those that are naturally occurring or modified in a manner that does not interfere with the purpose to which they are put. Modified bases include but are not limited to 7-deaza A and G, 7-deaza-8-aza A and G, and other heterocyclic moieties.

It will be recognized that fluorescent labels are not to be limited to single species organic molecules, but include inorganic molecules, multi-molecular mixtures of organic and/or inorganic molecules, crystals, heteropolymers, and the like. Thus, for example, CdSe—CdS core-shell nanocrystals enclosed in a silica shell can be easily derivatized for coupling to a biological molecule (Bruchez et al. (1998) Science, 281: 2013-2016). Similarly, highly fluorescent quantum dots (zinc sulfide-capped cadmium selenide) have been covalently coupled to biomolecules for use in ultrasensitive biological detection (Warren and Nie (1998) Science, 281: 2016-2018).

B) Polypeptide-Based Assays.

1) Assay Formats.

In addition to, or in alternative to, the detection of nucleic acid expression level(s), alterations in expression or activity of a an A2A receptor or other component of an A2A receptor signaling pathway can be detected and/or quantified by detecting and/or quantifying the amount and/or activity of a translated an A2A receptor protein or other component of an A2A receptor signaling pathway.

2) Detection of Expressed Protein

The A2A receptor or other component of an A2A receptor signaling pathway can be detected and quantified by any of a number of methods well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, western blotting, and the like.

In one preferred embodiment, an A2A receptor or other component of an A2A receptor signaling pathway is detected/quantified in an electrophoretic protein separation (e.g. a 1- or 2-dimensional electrophoresis). Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

In another preferred embodiment, Western blot (immunoblot) analysis is used to detect and quantify the presence of a an A2A receptor or other component of an A2A receptor signaling pathway. This technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the target polypeptide(s).

The antibodies specifically bind to the target polypeptide(s) and may be directly labeled or alternatively may be subsequently detected using labeled antibodies (e.g., labeled sheep anti-mouse antibodies) that specifically bind to a domain of the antibody.

In preferred embodiments, an A2A receptor or other component of an A2A receptor signaling pathway is detected using an immunoassay. As used herein, an immunoassay is an assay that utilizes an antibody to specifically bind to the analyte (e.g., the target polypeptide(s)). The immunoassay is thus characterized by detection of specific binding of a polypeptide of this invention to an antibody as opposed to the use of other physical or chemical properties to isolate, target, and quantify the analyte.

Any of a number of well recognized immunological binding assays (see, e.g., U.S. Pat. Nos. 4,366,241; 4,376,110; 4,517,288; and 4,837,168) are well suited to detection or quantification of the polypeptide(s) identified herein. For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

Immunological binding assays (or immunoassays) typically utilize a “capture agent” to specifically bind to and often immobilize the analyte (an A2A receptor protein). In preferred embodiments, the capture agent is an antibody.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled polypeptide or a labeled antibody that specifically recognizes the already bound target polypeptide. Alternatively, the labeling agent may be a third moiety, such as another antibody, that specifically binds to the capture agent/polypeptide complex.

Other proteins capable of specifically binding immunoglobulin constant regions, such as protein A or protein G may also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

Preferred immunoassays for detecting the target polypeptide(s) are either competitive or noncompetitive. Noncompetitive immunoassays are assays in which the amount of captured analyte is directly measured. In one preferred “sandwich” assay, for example, the capture agents (antibodies) can be bound directly to a solid substrate where they are immobilized. These immobilized antibodies then capture the target polypeptide present in the test sample. The target polypeptide thus immobilized is then bound by a labeling agent, such as a second antibody bearing a label.

In competitive assays, the amount of analyte (e.g., A2A receptor protein) present in the sample is measured indirectly by measuring the amount of an added (exogenous) analyte displaced (or competed away) from a capture agent (antibody) by the analyte present in the sample. In one competitive assay, a known amount of, in this case, labeled polypeptide is added to the sample and the sample is then contacted with a capture agent. The amount of labeled polypeptide bound to the antibody is inversely proportional to the concentration of target polypeptide present in the sample.

In one particularly preferred embodiment, the antibody is immobilized on a solid substrate. The amount of target polypeptide bound to the antibody may be determined either by measuring the amount of target polypeptide present in an polypeptide/antibody complex, or alternatively by measuring the amount of remaining uncomplexed polypeptide.

The immunoassay methods of the present invention include an enzyme immunoassay (EIA) which utilizes, depending on the particular protocol employed, unlabeled or labeled (e.g., enzyme-labeled) derivatives of polyclonal or monoclonal antibodies or antibody fragments or single-chain antibodies that bind beta/gammer dimer polypeptide(s), either alone or in combination. In the case where the antibody that binds the target polypeptide(s) is not labeled, a different detectable marker, for example, an enzyme-labeled antibody capable of binding to the monoclonal antibody which binds the target polypeptide, may be employed. Any of the known modifications of EIA, for example, enzyme-linked immunoabsorbent assay (ELISA), may also be employed. As indicated above, also contemplated by the present invention are immunoblotting immunoassay techniques such as western blotting employing an enzymatic detection system.

The immunoassay methods of the present invention may also be other known immunoassay methods, for example, fluorescent immunoassays using antibody conjugates or antigen conjugates of fluorescent substances such as fluorescein or rhodamine, latex agglutination with antibody-coated or antigen-coated latex particles, haemagglutination with antibody-coated or antigen-coated red blood corpuscles, and immunoassays employing an avidin-biotin or strepavidin-biotin detection systems, and the like.

The particular parameters employed in the immunoassays of the present invention can vary widely depending on various factors such as the concentration of antigen in the sample, the nature of the sample, the type of immunoassay employed and the like. Optimal conditions can be readily established by those of ordinary skill in the art. In certain embodiments, the amount of antibody that binds the target polypeptide(s) is typically selected to give 50% binding of detectable marker in the absence of sample. If purified antibody is used as the antibody source, the amount of antibody used per assay will generally range from about 1 ng to about 100 ng. Typical assay conditions include a temperature range of about 4° C. to about 45° C., preferably about 25° C. to about 37° C., and most preferably about 25° C., a pH value range of about 5 to 9, preferably about 7, and an ionic strength varying from that of distilled water to that of about 0.2M sodium chloride, preferably about that of 0.15M sodium chloride. Times will vary widely depending upon the nature of the assay, and generally range from about 0.1 minute to about 24 hours. A wide variety of buffers, for example PBS, may be employed, and other reagents such as salt to enhance ionic strength, proteins such as serum albumins, stabilizers, biocides and non-ionic detergents may also be included.

The assays of this invention are scored (as positive or negative or quantity of target polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of target polypeptide concentration.

Antibodies for use in the various immunoassays described herein are commercially available or can be produced using standard methods known to those of skill in the art.

C) Behavioral Assays.

In various embodiments behavioral assays can be used in place of or to supplement the assays for agents that alter expression or activity of an adenosine A2A receptor. Thus, for example, where a compound is already known to be an adenosine A2A receptor antagonist behavioral assays can be used to evaluate the compound for efficacy in inhibiting one or more components of behavior associated with addiction.

Such behavioral assays are well known to those of skill in the art. These include, but are not limited to operant self-administration (e.g., of ethanol or other substance), inhibition of adenosine-mediated reinstatement seeking behavior, etc. Several such assays are illustrated herein in the Examples and references cited therein.

D) Pre-Screening for Test Agents that Bind Adenosine A2A Receptors.

In certain embodiments it is desired to pre-screen test agents for the ability to interact with (e.g. specifically bind to) a nucleic acid that encodes an adenosine A2A receptor and/or to an adenosine A2A receptor. Specifically, binding test agents are likely to interact with and thereby inhibit A2A receptor expression and/or activity. Thus, in some preferred embodiments, the test agent(s) are pre-screened for binding to A2A receptor nucleic acids or to A2A receptors or A2A receptor proteins before performing the more complex assays described above.

In one embodiment, such pre-screening is accomplished with simple binding assays. Means of assaying for specific binding or the binding affinity of a particular ligand for a nucleic acid or for a protein are well known to those of skill in the art. In preferred binding assays, the target (e.g. an A2A receptor protein or nucleic acid) is immobilized and exposed to a test agent (which can be labeled), or alternatively, the test agent(s) are immobilized and exposed to an A2A receptor which can be labeled. The immobilized moiety is then washed to remove any unbound material and the bound test agent or bound receptor protein is detected (e.g. by detection of a label attached to the bound molecule). The amount of immobilized label is proportional to the degree of binding between the target and the test agent.

E) Scorin2 the Assay(s).

The assays of this invention are scored according to standard methods well known to those of skill in the art. The assays of this invention are typically scored as positive where there is a difference between the activity seen with the test agent present or where the test agent has been previously applied, and the (usually negative) control. In certain preferred embodiments, the change/difference is a statistically significant change/difference, e.g. as determined using any statistical test suited for the data set provided (e.g. t-test, analysis of variance (ANOVA), semiparametric techniques, non-parametric techniques (e.g. Wilcoxon Mann-Whitney Test, Wilcoxon Signed Ranks Test, Sign Test, Kruskal-Wallis Test, etc.). Preferably the difference/change is statistically significant at a greater than 80%, preferably greater than about 90%, more preferably greater than about 98%, and most preferably greater than about 99% confidence level. Most preferred “positive” assays show at least a 1.2 fold, preferably at least a 1.5 fold, more preferably at least a 2 fold, and most preferably at least a 4 fold or even a 10-fold difference from the negative control.

F) Agents for Screening: Combinatorial Libraries (e.g., Small Organic Molecules).

Virtually any agent can be screened according to the methods of this invention. Such agents include, but are not limited to nucleic acids, proteins, sugars, polysaccharides, glycoproteins, lipids, and small organic molecules. The term small organic molecule typically refers to molecules of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

Conventionally, new chemical entities with useful properties are generated by identifying a chemical compound (called a “lead compound”) with some desirable property or activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

In one preferred embodiment, high throughput screening methods involve providing a library containing a large number of potential therapeutic compounds (candidate compounds). Such “combinatorial chemical libraries” are then screened in one or more assays, as described herein to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis by combining a number of chemical “building blocks” such as reagents. For example, a linear combinatorial chemical library such as a polypeptide (e.g., mutein) library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, one commentator has observed that the systematic, combinatorial mixing of 100 interchangeable chemical building blocks results in the theoretical synthesis of 100 million tetrameric compounds or 10 billion pentameric compounds (Gallop et al. (1994) J. Med. Chem., 37(9): 1233-1250).

Preparation of combinatorial chemical libraries is well known to those of skill in the art. Such combinatorial chemical libraries include, but are not limited to, peptide libraries (see, e.g., U.S. Pat. No. 5,010,175, Furka (1991) Int. J. Pept. Prot. Res., 37: 487-493, Houghton et al. (1991) Nature, 354: 84-88). Peptide synthesis is by no means the only approach envisioned and intended for use with the present invention. Other chemistries for generating chemical diversity libraries can also be used. Such chemistries include, but are not limited to: peptoids (PCT Publication No WO 91/19735, 26 Dec. 1991), encoded peptides (PCT Publication WO 93/20242, 14 Oct. 1993), random bio-oligomers (PCT Publication WO 92/00091, 9 Jan. 1992), benzodiazepines (U.S. Pat. No. 5,288,514), diversomers such as hydantoins, benzodiazepines and dipeptides (Hobbs et al., (1993) Proc. Nat. Acad. Sci. USA 90: 6909-6913), vinylogous polypeptides (Hagihara et al. (1992) J. Amer. Chem. Soc. 114: 6568), nonpeptidal peptidomimetics with a Beta-D-Glucose scaffolding (Hirschmann et al., (1992) J. Amer. Chem. Soc. 114: 9217-9218), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) J. Org. Chem. 59: 658). See, generally, Gordon et al., (1994) J. Med. Chem. 37:1385, nucleic acid libraries (see, e.g., Strategene, Corp.), peptide nucleic acid libraries (see, e.g., U.S. Pat. No. 5,539,083) antibody libraries (see, e.g., Vaughn et al. (1996) Nature Biotechnology, 14(3): 309-314), and PCT/US96/10287), carbohydrate libraries (see, e.g., Liang et al. (1996) Science, 274: 1520-1522, and U.S. Pat. No. 5,593,853), and small organic molecule libraries (see, e.g., benzodiazepines, Baum (1993) C&EN, January 18, page 33, isoprenoids U.S. Pat. No. 5,569,588, thiazolidinones and metathiazanones U.S. Pat. No. 5,549,974, pyrrolidines U.S. Pat. Nos. 5,525,735 and 5,519,134, morpholino compounds U.S. Pat. No. 5,506,337, benzodiazepines U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include, but are not limited to, automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist and the Venture™ platform, an ultra-high-throughput synthesizer that can run between 576 and 9,600 simultaneous reactions from start to finish (see Advanced ChemTech, Inc. Louisville, Ky.)). Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

G) High Throughput Screening

Any of the assays for compounds modulating the accumulation or degradation of metabolic products described herein are amenable to high throughput screening. Preferred assays detect in adenosine A2A receptor expression or activity in response to the presence of a test compound.

The cells utilized in the methods of this invention need not be contacted with a single test agent at a time. To the contrary, to facilitate high-throughput screening, a single cell may be contacted by at least two, preferably by at least 5, more preferably by at least 10, and most preferably by at least 20 test compounds. If the cell scores positive, it can be subsequently tested with a subset of the test agents until the agents having the activity are identified.

High throughput assays for various reporter gene products are well known to those of skill in the art. For example, multi-well fluorimeters are commercially available (e.g., from Perkin-Elmer).

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols the various high throughput. Thus, for example, Zymark Corp. provides technical bulletins describing screening systems for detecting the modulation of gene transcription, ligand binding, and the like.

H) Modulator Databases.

In certain embodiments, the agents that score positively in the assays described herein (e.g. show an ability to inhibit A2A receptor expression or activity) can be entered into a database of putative and/or actual inhibitors of one or more components of addictive behavior associated with consumption of substance of abuse or to withdrawal therefrom. The term database refers to a means for recording and retrieving information. In preferred embodiments the database also provides means for sorting and/or searching the stored information. The database can comprise any convenient media including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems”, mainframe systems, distributed nodes on an inter- or intranet, data or databases stored in specialized hardware (e.g. in microchips), and the like.

### EXAMPLES

The following examples are offered to illustrate, but not to limit the claimed invention.

### Example 1

### Ethanol Operant Self-Administration in Rats is Regulated by Adenosine A2A Receptors

Dopamine (DA) release in the nucleus accumbens (NAc) is involved in neural and behavioral effects of ethanol (EtOH). Recent advances suggest that adenosine also plays an important role in regulating CNS responses to EtOH. Studies in neural cell culture show that EtOH, via activation of adenosine A₂ receptors (A₂), triggers cAMP/PKA signaling and CRE-mediated gene expression. Most importantly, subthreshold concentrations of EtOH acting through A₂, and a DA D₂ receptor (D2) agonist synergise to activate this pathway. Synergy is mediated by Gi/o βγ dimers (Yao et al., 2002). Recently, we reported that expression of a βγ inhibitor in the NAc reduces EtOH drinking in rats (Yao et al., 2002). If the rewarding effects of EtOH are mediated through this pathway, then A₂ or D₂ blockade should attenuate EtOH consumption.

Methods: Male Long Evans rats were trained to self-administer 10% EtOH in daily 30-min sessions with an active and inactive lever. Separate groups of rats were given the D₂ antagonist eticlopride (0.005, 0.007, 0.01 mg/kg), the A2A antagonist DMPX (1, 3, 5, 7, 10 and 20 mg/kg) and the Al antagonist DPCPX (0.125, 0.25, 0.5 mg/kg) by systemic injections.

Results: Eticlopride dose-dependently reduced EtOH drinking. DMPX showed a bimodal effect: 10 and 20 mg/kg decreased but 1 mg/kg increased EtOH consumption. DPCPX was without effect.

Conclusions: In support of our hypothesis, both D₂ and A2A antagonists attenuate EtOH self-administration. Low doses of an A2A antagonist enhance EtOH drinking, consistent with the possibility that rats increase EtOH self-administration to overcome partial A2A blockade. These data provide the first evidence that pharmacological modulation of the adenosine A2A receptors can regulate EtOH consumption in rats.

Introduction

The nucleus accumbens (NAc) and dorsal striatum express the highest concentrations of adenosine A2A receptors in the CNS (Jarvis and Williams, 1989; Svenningsson et al., 1997a). Experiments in neural cell culture systems have demonstrated that ethanol (EtOH) activates A₂ signaling (Gordon and Diamond, 1986). This occurs because EtOH blocks uptake of adenosine via an equilibrative nucleoside transporter, ENT1, causing an increase in extracellular adenosine concentrations (Nagy et al., 1990; Krauss et al., 1993). Increased extracellular adenosine activates A₂ receptors, resulting in increased cAMP levels. Ethanol-induced increases in cAMP lead to the activation of PKA and translocation of the catalytic subunit of PKA (PKA Cα) to the nucleus (Dohrman et al., 2002). This is followed by increases in cAMP-dependent CRE-mediated gene transcription (Yao et al., 2002).

In addition to the possibility of EtOH increasing extracellular adenosine by inhibiting adenosine uptake, EtOH metabolism in the liver can also lead to increases in adenosine in tissues and organs (Carmichael et al., 1987, 1988, 1991; Orrego et al., 1988a). Hepatic alcohol and acetaldehyde activity generates acetate from EtOH (Orrego et al., 1988b). Acetate is further metabolized to acetyl CoA consuming ATP in the process; this generates adenosine (Israel et al., 1994). Adenosine released into the circulation crosses the blood-brain barrier (Cornford and Oldendorf, 1975). In addition, acetate generated by alcohol metabolism in the liver is released into the circulation where it also crosses the blood-brain barrier. Acetate entering the brain is readily converted to acetyl CoA, (Berl and Frigyesi, 1969). This would generate adenosine in situ. Acetate, like adenosine, is a CNS depressant. The effect of acetate in the brain appears to be mediated by adenosine because it is blocked by adenosine receptor antagonists (Israel et al., 1994; Campisi et al., 1997). Taken together, these findings suggest that some of the behavioral effects of EtOH in vivo may be mediated by both direct and indirect EtOH-induced increases in extracellular adenosine in the brain with subsequent activation of adenosine receptors.

One of the unique characteristics of the NAc and dorsal striatum is the co-expression of adenosine A2A and dopamine (DA) D₂ receptors on the same GABAergic medium spiny neurons (Fink et al., 1992). We have examined the interaction of A₂ and D₂ receptors in mediating neuronal responses to EtOH in cell culture systems. We discovered a synergy between D₂ and EtOH/A₂-induced activation. Subthreshold concentrations of a D₂ agonist or EtOH, which have no effect alone, when added together induced maximal activation of PKA signaling. Moreover, we found that release of Gi/o βγ dimers is required for synergy induced by D₂ agonists and EtOH. In support of our model, we found that inhibition of βγ dimer action in the shell region of the NAc decreases EtOH self-administration (Yao et al., 2002). Because A2A receptors functionally interact with D₂ receptors within the same striatal GABAergic medium spiny neurons of the NAc (Fink et al., 1992; Ferre, 1997; Ferre et al., 1997; Fuxe et al., 1998; Svenningsson et al., 1999), we hypothesize that synergy between A2A and D₂ receptors confers selective EtOH hypersensitivity to this brain region. Specifically, we suggest that, in vivo, EtOH itself contributes to this synergistic interaction both by increasing firing of VTA DA neurons (Appel et al., 2003) and hence enhancing DA levels in the NAc (Imperato and Di Chiara, 1986; Weiss et al., 1993), and by increasing extracellular adenosine via the mechanisms discussed above. The reinforcing effects of EtOH may be mediated by these EtOH-induced increases in both DA and adenosine acting upon D₂ and A2A receptors, respectively.

The studies in this paper examine whether blockade of D₂ or A2A receptors reduce EtOH's reinforcing effects. We used the A2A antagonist DMPX to determine whether adenosine A2A blockade would reduce EtOH self-administration in rats. To rule out the possible involvement of the adenosine A₁ receptor during EtOH consumption, the Al selective antagonist DPCPX was also tested. D₂ receptors have already been shown to regulate EtOH self-administration (Hodge et al., 1997; Cohen et al., 1998; Czachowski et al., 2001). To further characterize the participation of D₂ receptors under our conditions of study, we tested the effects of the D₂ antagonist eticlopride, which to our knowledge has not been tested in studies of EtOH operant self-administration in rats.

Methods

Animals and Housing

Male Long Evans rats (Harlan, Indianapolis, Ind.) weighing approximately 250 g at the beginning of the studies, were individually housed with food and water available ad libitum except when stated otherwise. They were maintained on a 12 h light/dark cycle, lights on at 7:00 am. Operant training occurred between 8:30 am and 2:00 pm. The experimental procedures were approved in advance by our Institutional Animal Care and Use Committee.

Drugs

EtOH dilution (10% v/v) for self-administration was made up using 95% ethyl alcohol and tap water. Sucrose (Saccharose, Fisher Scientific, Fair Lawn, N.J., USA) solution (10% w/v) was made up with tap water. All the compounds tested were obtained from Sigma Chemical Co. (St. Louis, Mo.). The A2A antagonist DMPX (3,7-Dimethyl-1-propargylxanthine) was dissolved in warm saline. The A1 antagonist DPCPX (8-Cyclopentyl-1,3-dipropylxanthine) was dissolved in 20:80 v/v mixture of Alkamuls EL-620 (Rhodia Inc., Cranbury, N.J., USA) and phosphate buffered saline. The D₂ antagonist, eticlopride, was dissolved in saline. Drugs were administered in a 1 ml/kg volume except for the group tested with 5, 7 and 20 mg/kg DMPX, in which the injection volume was 2 ml/kg as the highest DMPX soluble concentration achieved was 10 mg/ml. Drugs were prepared fresh every treatment day.

Operant Self-Administration Apparatus

EtOH operant self-administration was carried out in standard operant chambers (Med Associates, Georgia, Vt.) housed in sound-attenuated cubicles. Each chamber (33×30.5×33 cm) contained two retractable levers against the right wall, 7 cm from the floor and 1 cm from the right or left edge of the right wall, respectively. One recessed dish positioned at 2.5 cm above floor level and 6 cm from the levers towards the center of the chamber was the reinforcer receptacle. Fluid (0.1 ml) was delivered from syringe pumps upon activation of 1 of the 2 retractable response levers. A 3 sec tone was activated upon lever pressing. Pressing the inactive lever resulted in no visual/auditory cue or reinforcement delivery, except during sucrose overnight sessions (see below). The beginning of a training session was signaled by the onset of the house light located in the center of the wall facing the levers, at 27.2 cm above the floor. A computer controlled stimulus and fluid delivery and recorded operant responses.

EtOH Self-Administration Procedure

Before the beginning of EtOH operant self-administration, rats were exposed to a 10% EtOH (1OE) solution as the only liquid source in their home cages for 4 days. For the next 14 days, animals were allowed free choice between 10E in tap water or tap water from graded glass tubes. At the end of this 14-day period, operant self-administration was initiated according to the sucrose fading technique (Samson, 1986) with minor modifications. Rats were restricted to 30 minutes of water per day for 2 consecutive days. On the night of the second day of water restriction, rats were placed in the operant chambers for a 12-15 hrs overnight session on an FR1 schedule (1 reinforcement of 0.1 ml per lever press) with 10% sucrose (10S) as reinforcer and both levers active. The next day, rats began the operant self-administration training. Animals were kept on water restriction for the next 4-5 days during which they received one 45 min session per day on an FR1 schedule with 10S as reinforcer and one active lever. They were then given free water in their home cages for the remainder of the experiment and were trained for 2-3 more of the above described sessions. The next day, sessions were shortened to 30 minutes and the ratio of responding was increased to FR3. EtOH was added to the sweet solution (10S10E) and rats received 3-4 sessions of this solution, followed by at least 20 sessions with 10E only. A minimum average of 0.3 g/kg EtOH consumption in 8 sessions prior to the beginning of any drug treatment was required. Animals that failed to consume this average amount of EtOH in the last 8 sessions were not included in the study.

Experimental Design

Once rats achieved stable responding for EtOH, they were habituated to subcutaneous (sc) or intraperitoneal (ip) injections of vehicle during one session per week for 2 consecutive weeks. Next, drugs were tested using a within-subjects Latin Square design, whereby each animal received each dose of one of the compounds and the appropriate vehicle. Test sessions took place Wednesday or Thursday of each week although rats were trained everyday from Monday through Friday. The three drugs used in this study were tested in 4 separate groups of animals. DMPX (0, 1, 3, 5, 7, 10 and 20 mg/kg) or vehicle was administered ip 20 minutes prior to each session. The doses of 1, 3 and 10 mg/kg DMPX were tested in one group of animals while the remaining DMPX doses (0, 5, 7 and 20 mg/kg) were studied in a different group to better examine DMPX concentrations around the significant 10 mg/kg dose. DPCPX (0, 0.125, 0.25 or 0.5 mg/kg) or vehicle was injected ip 15 minutes prior to each session. Eticlopride (0, 0.005, 0.007 or 0.01 mg/kg) or vehicle was administered sc 25 minutes prior to each session.

Statistics

Number of lever presses, number of EtOH reinforcements, g/kg of EtOH consumption as well as number of inactive lever presses per session was analyzed by one-way ANOVA with within subject factors being DMPX, DPCPX or eticlopride doses. For the DMPX effect, the group of animals tested with 1,3 and 10 mg/kg was analyzed separately from the animals receiving 5, 7 and 20 mg/kg DMPX. Post-hoc LSD tests were performed where appropriate.

Results

Effect of DMPX on EtOH Self-Administration

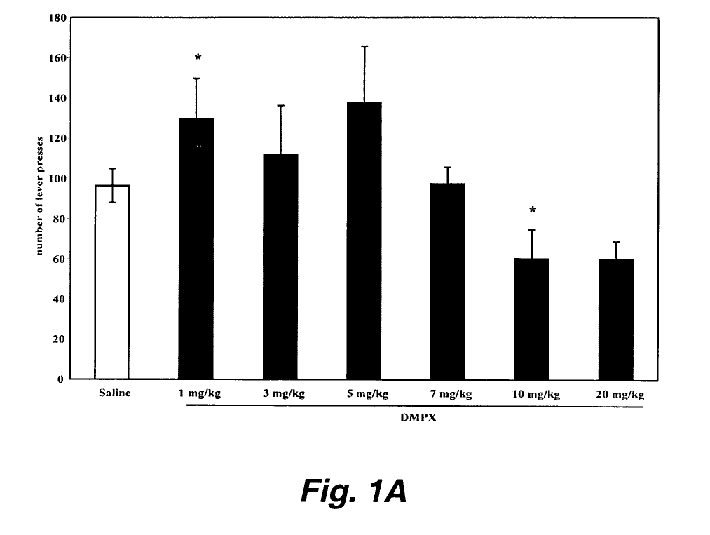

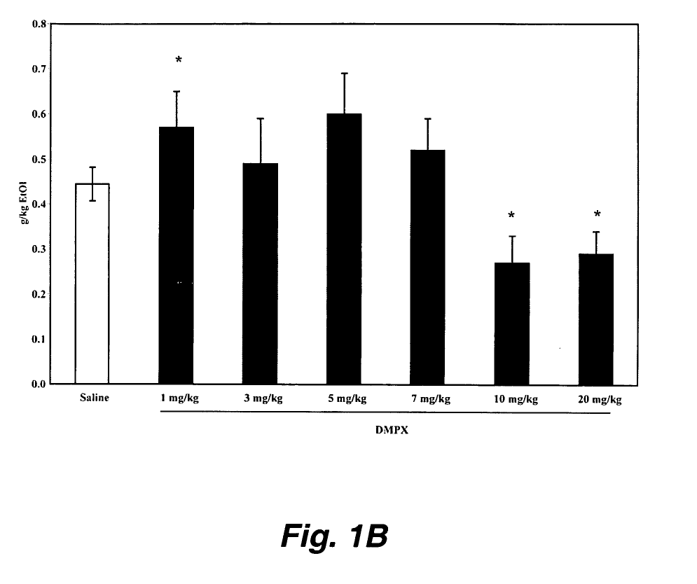

Although 2 separate dose-effect functions were determined for DMPX in 2 separate groups of rats, the results for the effects of all doses of DMPX tested are shown in one figure for clearer comparison (FIG. 1). Mean responding following saline treatment was not different: Group 1: 89.20±15.26 (0.39±0.06 g/kg), and Group 2: 99.44+12.02 (0.46+0.05 g/kg). In the group tested with 1, 3 and 10 mg/kg DMPX, significant effects of number of lever presses [F(3,27)=9.68, p<0.0003], number of EtOH reinforcements [F(3,27)=8.69, p<0.0003] and g/kg EtOH consumption [F(3,27)=8.62, p<0.0004] observed. Inactive lever presses were not affected by any of these doses [F(3,27)=0.87, NS] (Table 1). The group tested with 5, 7 and 20 mg/kg DMPX showed significant effects of number of lever presses [F(3,21)=4.37, p<0.02], number of EtOH reinforcements [F(3,21)=4.76, p<0.02] and g/kg EtOH consumption [F(3,21)=4.96, p<0.01], but n significant effect of number of inactive lever presses was observed [F(3,21)=0.15, NS] (Table 1). The dose-effect function of the A2A antagonist was bimodal, as revealed by post-hoc tests. The lowest dose tested (1 mg/kg) significantly increased the number of lever presses (p<0.02), the number of EtOH reinforcements (p<0.03) as well as g/kg of EtOH intake (p<0.03). The middle doses, 3, 5 and 7 mg/kg did not significantly affect any of the measures. The dose of 10 mg/kg significantly decreased all measures analyzed: number of lever presses (p<0.02), number of EtOH reinforcements (p<0.03) as well as g/kg of EtOH consumption (p<0.02). The highest dose (20 mg/kg) showed a significant effect on number EtOH reinforcements (p<0.05) and g/kg EtOH intake (p<0.05).

TABLE 1
Effect of DMPX, DPCPX and eticlopride on number of inactive lever presses.
DMPX

| Saline | 1 mg/kg | 3 mg/kg | 5 mg/kg | 7 mg/kg | 10 mg/kg | 20 mg/kg |
|---|---|---|---|---|---|---|
| n = 18 | n = 10 | n = 10 | n = 8 | n = 8 | n = 10 | n = 8 |
| 2.92 ± 0.45 | 2.60 ± 0.85 | 1.50 ± 0.37 | 3.56 ± 1.29 | 3.93 ± 2.17 | 3.60 ± 1.76 | 3.87 ± 0.64 |

DPCPX

| Vehicle | 0.125 mg/kg | 0.25 mg/kg | 0.5 mg/kg |
|---|---|---|---|
| n = 7 | n = 7 | n = 7 | n = 7 |
| 2.71 ± 0.56 | 1.78 ± 0.62 | 2.36 ± 1.08 | 2.86 ± 0.88 |

ETICLOPRIDE

| Saline | 0.005 mg/kg | 0.007 mg/kg | 0.01 mg/kg |
|---|---|---|---|
| n = 5 | n = 5 | n = 5 | n = 5 |
| 2.00 ± 0.67 | 2.00 ± 0.58 | 1.00 ± 0.41 | 1.83 ± 0.98 |

Values are given as mean ± SEM

Effect of DPCPX on EtOH Self-Administration

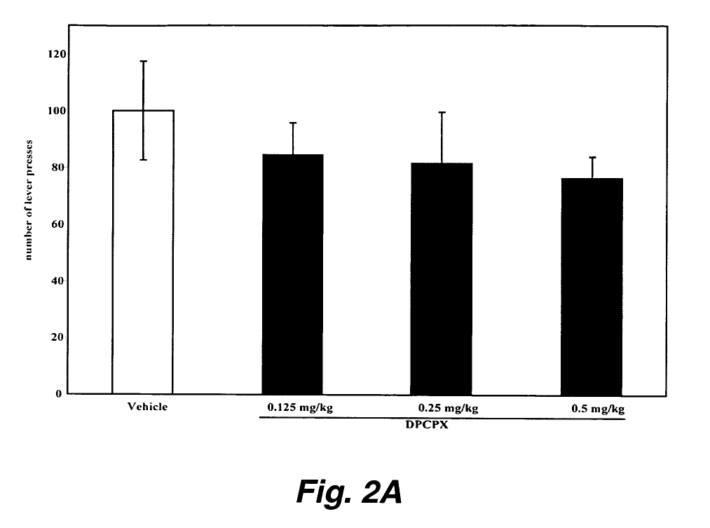

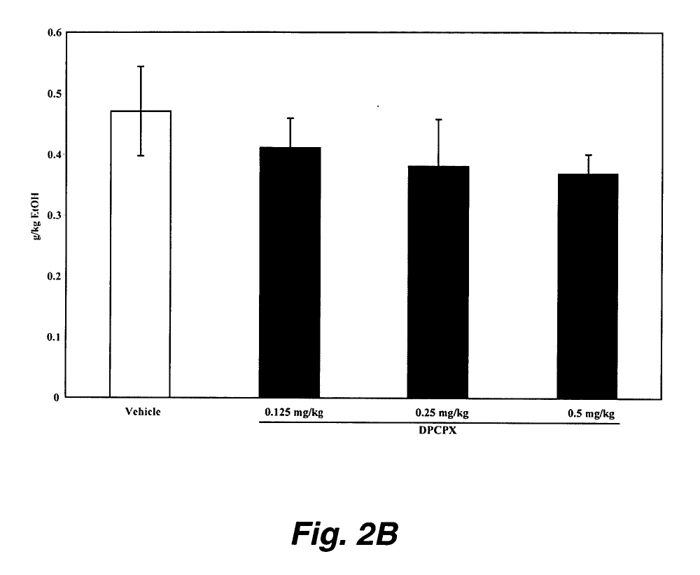

Results of this experiment are shown in FIG. 2 and Table 1. There was no effect of the selective A₁ antagonist, DPCPX (0.125, 0.25, or 0.5 mg/kg), on any of the parameters measured: number of lever presses [F(3,18)=0.84, NS], number of EtOH reinforcements [F(3,18)=0.74, NS], g/kg of EtOH consumption [F(3,18)=0.84, NS] or inactive lever presses [F(3,18)=0.52, NS].

Effect of Eticlopride on EtOH Self-Administration

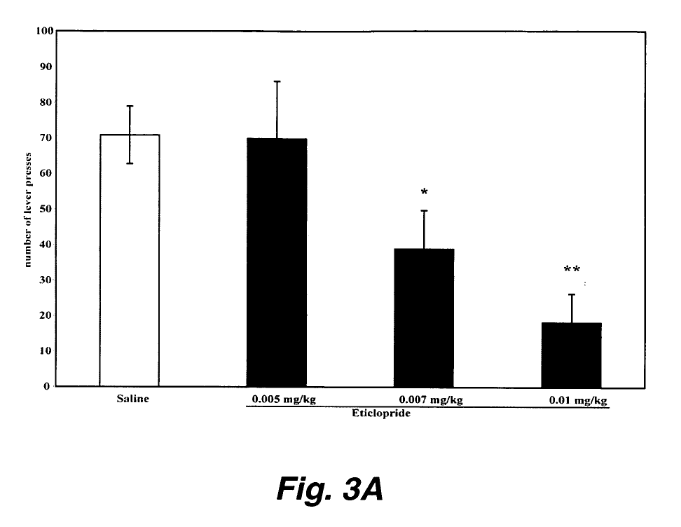

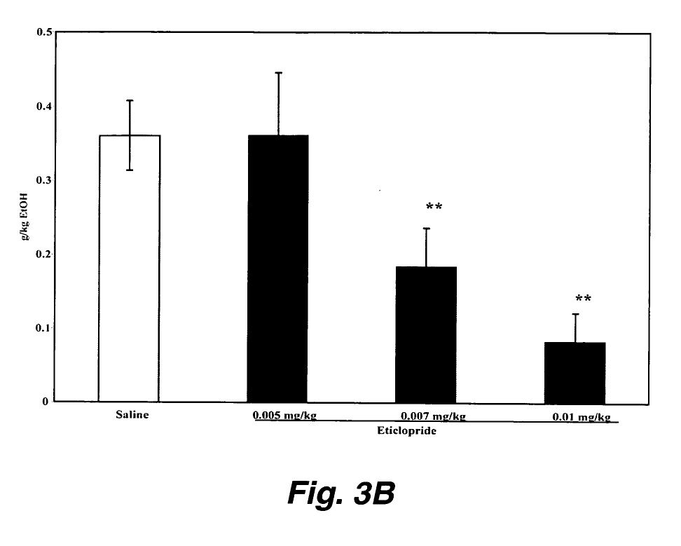

Results of this experiment are shown in FIG. 3. A significant decrease in the 10 number of lever presses [F(3,15)=11.13, p<0.0004], the number of EtOH reinforcements [F(3,15)=8.96, p<0.001] as well as g/kg of EtOH consumption [F(3,15) <10.14, p<0.0007] was observed. Post-hoc analyses revealed significant effects of 0.007 mg/kg and 0.01 mg/kg eticlopride on all the measurements analyzed: number of lever presses (p<0.005 and p<0.0001, respectively), number of reinforcements (p<0.02 and p<0.007, respectively) and g/kg of EtOH consumption (p<0.01 and p<0.004, respectively). The dose of 0.005 mg/kg did not significantly affect any of the analyzed measurements. Inactive lever presses were not affected by any of the doses [F(3,15)=0.39, NS] (Table 1).

Discussion

The major finding in this study is that adenosine A2A receptors regulate the reinforcing properties of EtOH. The A2A antagonist, DMPX, bimodally affected the number of lever presses, number of reinforcements, and g/kg of EtOH consumed during operant self-administration. By contrast, there was no effect of an adenosine Al antagonist. As expected, the DA D₂ antagonist, eticlopride, decreased all the parameters measured, as reported with other D₂ antagonists (Hodge et al., 1997; Cohen et al., 1998; Czachowski et al., 2001).

EtOH inhibits adenosine re-uptake via the EtOH-sensitive equilibrative nucleoside transporter, ENT-1 (Nagy et al., 1990; Handa et al., 2001) leading to an increase in extracellular adenosine. Adenosine activates A₂ receptors and increases cAMP/PKA signaling in cell culture (Gordon et al., 1986). Recently, we reported synergy between NPA, a D₂ agonist and EtOHI/A₂ for PKA activation. Synergy is mediated by βγ dimers released from Gi/o (Yao et al., 2002). We have also provided support for a role of this pathway in EtOH's behavioral effects in vivo: inhibition of βγ dimers in rat NAc neurons decreases EtOH self-administration (Yao et al., 2002). The current study provides further support for the importance of A2A and D₂ in this process by demonstrating that A2A or D₂ receptor blockade decreases EtOH self-administration in vivo.

We found that systemic administration of DMPX (10 and 20 mg/kg) decreased EtOH self-administration. However, the lowest dose of DMPX paradoxically increased the number of lever presses and, consequently, EtOH consumption. There are several possible explanations for this biphasic effect of DMPX. First, low and high doses of DMPX may involve actions at distinct receptor populations; for example, at low doses DMPX may only inhibit high affinity A₂ receptors, and at higher doses, DMPX may inhibit both high and low affinity receptors (Sebastiao and Ribero, 1992; Cunha et al., 1999; El Yacoubi et al., 2000). It is conceivable that DMPX can also bind to A₁ receptors, although with less selectivity (Jacobson et al., 1993). Therefore, the effects of DMPX at higher doses could have also involved A₁ receptors. Because of this possibility, we tested the effect of a selective A₁ antagonist on operant EtOH self-administration. None of the doses of the A₁ antagonist, DPCPX, affected any of the parameters measured so we think this explanation unlikely. A final possibility is that the A2A antagonist at low doses only partially blocks A2A receptors, leading to an increase in EtOH consumption to compensate for the decreased effectiveness of EtOH under these conditions. This phenomenon has been observed in animals self-administering morphine in the presence of lower doses of opiate antagonists (Koob et al., 1986). It appears that subjects will strive to overcome a partial blockade of receptors directly involved in self-administration, but when the same receptors are completely blocked at higher doses, subjects then reduce self-administration. This phenomenon provides additional behavioral pharmacological evidence that A2A receptors appear to be a direct and important mediator of EtOH self-administration.

Because adenosine receptors also affect locomotor activity in rodents (Seale et al., 1988; Nikodijevic et al., 1991; Barraco et al., 1993; Svenningsson et al., 1997b; Green and Schenk, 2002), it is possible that the effects of DMPX in the current study are due to its effects on locomotor activity and to effects on the reinforcing properties of EtOH. This does not appear to be a likely explanation because DMPX, like other A2A adenosine antagonists, increases locomotor activity; we found no effect of DMPX on the number of inactive lever responses, which is an indirect measure of locomotor activity.

The EtOH withdrawal syndrome and EtOH-induced motor incoordination appears, in part, to involve the adenosine system, mediated primarily through Al receptors (Malec et al., 1996; Jarvis and Becker, 1998; Barwick and Dar, 1998; Gatch et al., 1999; Kaplan et al., 1999; Dar, 2001). We find that Al receptors do not modulate EtOH self-administration. Two recent reports implicate the adenosine A2A receptor in CNS responses to EtOH. El Yacoubi et al. (2001) showed that the absence of or chronic blockade of A2A reduces handling-induced convulsions during EtOH withdrawal. Naassila et al. (2002) reported that A2A knock-out male mice consumed larger amounts of 6% and 20% but not 10% EtOH, while females consumed larger amounts of 6% and 10% but not 20% EtOH in a 2 bottle choice study when compared with wild type animals. By contrast, we find that adenosine A2A receptor blockade decreases EtOH consumption. The reasons for this discrepancy remain to be determined, but are likely due to adaptations and compensations in the A₂ knock-out mice, including the development of anxiety (Ledent et al., 1997) and possible changes in D₂ function because of the close physical association of D₂ with A₂ receptors (Franco et al., 2000).

Eticlopride, a potent D₂ antagonist, dose-dependently decreased the number of lever presses for EtOH as well as g/kg of EtOH consumed. There was no effect on the number of inactive lever presses. These results agree with several reports that other D₂ antagonists decrease EtOH consumption (Cohen et al., 1998; Czachowski et al., 2001; George et al., 1995; Hodge et al., 1997; Samson et al., 1993; Weiss et al., 1990). To our knowledge, however, this is the first study of eticlopride in EtOH operant self-administration. Taken together, these findings confirm the importance of D₂ in EtOH's rewarding properties. Our findings are also consistent with reports that EtOH self-administration elevates extracellular DA in NAc (Doyon et al., 2003; Weiss and Porrino, 2002; Weiss et al., 1993, 1996). Also, rats directly self-administer EtOH into the VTA (Gatto et al., 1994), and pharmacological manipulations of DA neurotransmission modifies EtOH-reinforced operant behavior and EtOH preference (Weiss et al., 1990; Samson et al., 1993; George et al., 1995; Hodge et al., 1997; Cohen et al., 1998; Czachowski et al., 2001).

In summary, we find that blockade of either A2A or D₂ receptors decreases EtOH self-administration. These findings support a role for endogenous adenosine and DA in the reinforcing effects of EtOH although it is not known whether the self-administered amounts of EtOH in the current studies were sufficient to cause increases in extracellular levels of either adenosine or DA. Despite this limitation, our results support a working model in which self-administered EtOH blocks the adenosine transporter potentiating an increase in extracellular adenosine via EtOH metabolism in the liver; this adenosine acts through A2A receptors to increase cAMP/PKA signaling in the NAc. Likewise, it appears that EtOH-stimulated DA release may also activate this PKA signaling cascade via D₂ receptors. We suggest that A₂ and D₂ receptor blockade reduces the reinforcing effects of EtOH by preventing these EtOH-induced effects. Studies are underway to determine whether synergy between A2A and D₂ receptors contributes to EtOH self-administration in vivo. To our knowledge, these data provide the first evidence that pharmacological manipulations of the adenosine A2A receptor in vivo can regulate EtOH consumption in rats. It is possible that drugs which block A2A receptor function might be beneficial in the treatment and prevention of excessive drinking in alcoholism.

### REFERENCES

Appel S B, Liu Z, McElvain M A, Brodie M S (2003) Ethanol excitation of dopaminergic ventral tegmental area neurons is blocked by quinidine. J Pharmacol Exp Ther 306:437-446.

Barraco R A, Martens K A, Pareizon M, Normile H J (1993) Adenosine A2A receptors in the nucleus accumbens mediate locomotor depression. Brain Res Bull 31:397-404.

Barwick V S, Dar M S (1998) Adenosinergic modulation of ethanol-induced motor incoordination in the rat motor cortex. Prog Neuro-Psychopharmacol & Biol Psychiat 22:587-607.

Berl S, Frigyesi T L (1969) The turnover of glutamate, glutamine, aspartate and GABA labeled with [1-14C]acetate in caudate nucleus, thalamus and motor cortex (CAT). Brain Res 12:444-455.

Campisi P, Carmichael F J, Crawford M, Orrego H, Khanna J M (1997) Role of adenosine in the ethanol-induced potentiation of the effects of general anesthetics in rats. Eur J Pharmacol 325:165-172.

Carmichael F J, Israel Y, Crawford M, Minhas K, Saldivia V, Sandrin S, Campisi P, Orrego H (1991) Central nervous system effects of acetate: Contribution to the central effects of ethanol. J Pharm Exp Ther 259:403-408.

Carmichael F J, Israel Y, Saldivia V, Giles H G, Meggiorini S, Orrego H (1987) Blood acetaldehyde and the ethanol-induced increase in splanchnic circulation. Biochem Pharmacol 36:2673-2678.

Carmichael F J, Saldivia V, Varghese G A, Israel Y, Orrego H (1988) Ethanol-induced increase in portal blood flow: role of acetate and A1- and A2-adenosine receptors. Amer J Physiol 255:G417-G423.

Cohen C, Perrault G, Sanger D J (1998) Preferential involvement of D3 versus D2 dopamine receptors in the effects of dopamine receptor ligands on oral ethanol self-administration. Psychopharmacol 140:478-485.

Comford E M, Oldendorf W H (1975) Independent blood-brain barrier transport systems for nucleic acid precursors. Biochim Biophys Acta 394, 211-219.

Cunha R A, Constantino M D, Ribeiro J A (1999) G protein coupling of CGS 21680 binding sites in the rat hippocampus and cortex is different from that of adenosine Al and striatal A2A receptors. Naunyn-Schmiedeberg's Arch Pharmacol 359:295-302.

Czachowski C L, Chappell A M, Samson H H (2001) Effects of raclopride in the nucleus accumbens on ethanol seeking and consumption. Alcoholism: Clin and Exp Res 25:1431-1440.

Dar M S (2001) Modulation of ethanol-induced motor incoordination by mouse striatal A₁ adenosinergic receptor. Brain Res Bull 55:513-520.

Dohrman D P, Chen H-M, Gordon A S, Diamond 1 (2002) Ethanol-induced translocation of PKA occurs in two phases: control by different molecular mechanisms. Alcohol Clin Exp Res 26:407-415.

Doyon W M, York J L, Diaz L M, Samson H H, Czachowski C L, Gonzales R A (2003) Dopamine activity in the nucleus accumbens during consummatory phases of oral ethanol self-administration. Alcohol Clin Exp Res 27:1573-1582.

El Yacoubi M, Ledent C, Parmentier M, Costentin J, Vaugeois J-M (2000) SCH 58261 and ZM 241385 differentially prevent the motor effects of CGS 21680 in mice: evidence for a functional ‘atypical’ adenosine A2A receptor. Eur J Pharm 401:63-77.

El Yacoubi M, Ledent C, Parmentier M, Daoust M, Costentin J, Vaugeois J-M (2001) Absence of the adenosine A2A receptor or its chronic blockade decrease ethanol withdrawal-induced seizures in mice. Neuropharmacol 40:424-432.

Ferre S (1997) Adenosine-dopamine interactions in the ventral striatum. Implications for treatment of schizophrenia. Psychopharmacology 133:107-120.

Ferre S, Fredholm B B, Morelli M, Popoli P, Fuxe K (1997) Adenosine-dopamine receptor-receptor interactions as an integrative mechanism in the basal ganglia. Trends in Neurosci 20:482-487.

Fink J S, Weaver D R, Rivkees S A, Peterfreund R A, Pollack A E, Adler E M, Reppert S M (1992) Molecular cloning of the rat A2 adenosine receptor: selective co-expression with D2 dopamine receptors in rat striatum. Mol Brain Res 14:186-195.

Franco R, Ferre S, Agnati L, Torvinen M, Gines S, Hillion J, Casado V, Lledo P, Zoli M, Lluis C, Fuxe K (2000) Evidence for adenosine/dopamine receptor interactions: indications for heteromerization. Neuropsychopharnacology 23:S50-59.

Fuxe K, Ferre S, Zoli M, Agnati L F (1998) Integrated events in central dopamine transmission as analyzed at multiple levels. Evidence for intramembrane adenosine A2A/doparine D₂ and adenosine A₁/dopamine D₁ receptor interactions in the basal ganglia. Brain Res Rev 26:258-273.

Gatch M B, Wallis C J, Lal H (1999) The effects of adenosine ligands R-PIA and CPT on ethanol withdrawal. Alcohol 19:9-14.

Gatto G J, McBride W J, Murphy J M, Lumeng L, Li T K (1994) Ethanol self infusion into the ventral tegmental area by alcohol preferring rats. Alcohol 11:557-564.

George S R, Fan T, Ng G Y, Jung S Y, O'Dowd B F, Naranjo C A (1995) Low endogenous dopamine function in brain predisposes to high alcohol preference and consumption: reversal by increasing synaptic dopamine. J Pharmacol Exp Ther 273:373-379.

Gordon A S, Collier K, Diamond I (1986) Ethanol regulations of adenosine receptor-stimulated cAMP levels in a clonal neural cell line: an in vitro model of cellular tolerance to ethanol. Proc Natl Acad Sci USA 83:2105-8.

Gordon A S, Diamond I (1986) Phosphorylation of the nicotinic acetylcholine receptor. Prog in Brain Res 69:141-148.

Green T A, Schenk S (2002) Dopaminergic mechanisms for caffeine-produced cocaine seeking in rats. Neuropsychopharmacol 26:422-430.

Handa M, Choi D S, Caldeiro R M, Messing R O, Gordon A S, Diamond I (2001) Cloning of a novel isoform of the mouse NBMPR-sensitive equilibrative nucleoside transporter (ENT1) lacking a putative phosphorylation site. Gene 262:301-7.

Hodge C W, Samson H H, Chapelle A M (1997) Alcohol self-administration: further examination of the role of dopamine receptors in the nucleus accumbens. Alcohol Clin Exp Res 21:1083-1091.

Imperato A, Di Chiara G (1986) Preferential stimulation of dopamine release in the nucleus accumbens of freely moving rats by ethanol. J Pharmacol Exp Ther 239:219-228.

Israel Y, Orrego H, Carmichael F J (1994) Acetate-mediated effects of ethanol. Alcohol Clin Exp Res 18:144-148.

Jacobson K A, Nikodijević O, Padgett W L, Gallo-Rodriguez C, Maillard M, Daly J W (1993) 8-(3-Chlorostyryl)caffeine (CSC) is a selective A2-adenosine antagonist in vitro and in vivo. FEBS 323:141-144.

Jarvis M F, Becker H C (1998) Single and repeated episodes of ethanol withdrawal increase adenosine A₁, but not A2A, receptor density in mouse brain. Brain Res 786:80-88.

Jarvis M F, Williams M (1989) Direct autoradiographic localization of adenosine A2 receptors in the rat brain using the A2-selective agonist [3H] CGS21680. Eur J Pharmacol 168:243-6.

Kaplan G B, Bharmal N H, Leite-Morris K A, Adams W R (1999) Role of adenosine A₁ and A₂ receptors in the alcohol withdrawal syndrome. Alcohol 19:157-162.

Krauss S W, Ghimikar R B, Diamond I, Gordon A S (1993) Inhibition of adenosine uptake by ethanol is specific for one class of nucleoside transporters. Mol Pharmcol 44:1021-6.

Koob G F, Vaccarino F J, Amalric M, Swerdlow N R (1986) Neural Substrates for Cocaine and Opiate Reinforcement, in Cocaine: Clinical and Biobehavioral aspects (Fisher S, Raskin A, Uhlenhuth E H eds), pp 80-108. Oxford University Press, Inc., New York.

Ledent C, Vaugeois J-M, Schiffmann S N, Pedrazzin T, El Yacoubi M, Vanderhaegen J-J, Costentin J, Heath J K, Vassart G, Parmentier M (1997) Aggressiveness, hypoalgesia and high blood pressure in mice lacking the adenosine A2A receptor. Nature 388: 674-678.

Malec D, Michalska E, Pikulicka J (1996) Influence of adenosinergic drugs on ethanol withdrawal syndrome in rats. Pol J Pharmacol 48:583-8.

Naassila M, Ledent C, Daoust M (2002) Low ethanol sensitivity and increased ethanol consumption in mice lacking adenosine A2A receptors. J Neurosci 22:10487-10493.

Nagy L E, Diamond I, Casso D J, Franklin C, Gordon A S (1990) Ethanol increases extracellular adenosine by inhibiting adenosine uptake via the nucleoside transporter. Journal of Biological Chemistry 265:1946-1951.

Nikodijevic O, Sarges R, Daly J W, Jacobson K A (1991) Behavioral effects of A1- and A2-selective adenosine agonists and antagonists: Evidence for synergism and antagonism. J Pharmacol Exp Ther 259:286-294.

Orrego H, Carmichael F J, and Israel Y (1988a) New insights on the mechanism of the alcohol-induced increase in portal blood flow. Can J Pharmacol 66:1-9.

Orrego H, Carmichael F J, Saldivia V, Giles H G, Sandrin S, and Israel Y (1988b) Ethanol-induced increase in portal blood flow: role of adenosine. Amer Physiol Soc G495-G501.

Samson H H (1986) Initiation of ethanol reinforcement using a sucrose-substitution procedure in food- and water-sated rats. Alcohol Clin Exp Res 10:436-442.

Samson H H, Hodge C W, Tolliver G A, Haraguchi M (1993) Effect of dopamine agonists and antagonists on ethanol reinforced behavior: the involvement of the nucleus accumbens. Brain Res Bull 30:133-141.

Seale T W, Abla K A, Shamim M T, Carney J M, Daly J W (1988) 3,7-Dimethyl-1-propargylxanthine: A potent and selective in vivo antagonist of adenosine analogs. Life Sciences (43):1671-1684.

Sebastiao A M, Ribeiro J A (1992) Adenosine A₂ receptor-mediated excitatory actions on the nervous system. Prog Neurobiol 48:167-189.

Svenningsson P, Hall H, Sedvall G and Fredholm BB (1997a) Distribution of adenosine receptors in the postmortem human brain: an extended autoradiographic study. Synapse 27:322-335.

Svenningsson P, Le Moine C, Fisone G, Fredholm B B (1999) Distribution, biochemistry and function of striatal adenosine A2A receptors. Progress in Neurobiology 59:355-396.

Svenningsson P, Nomiko G G, Ongini E, Fredholm B B (1997b) Antagonism of adenosine A2A receptors underlies the behavioural activating effect of caffeine and is associated with reduced expression of messenger RNA for NGFI-A and NGFI-B in caudate-putamen and nucleus accumbens. Neuroscience 79:753-764.

Weiss F, Lorang M T, Bloom F E, Koob G F (1993) Oral alcohol self-administration stimulates dopamine release in the rat nucleus accumbens: genetic and motivational determinants. J Pharmacol Exp Ther 267:250-258.

Weiss F, Mitchiner M, Bloom F E, Koob G F (1990) Free-choice responding for ethanol versus water in alcohol preferring (P) and unselected Wistar rats is differentially modified by naloxone, bromocriptine and methysergide. Psychopharmacology (Berl) 101:178-186.

Weiss F, Parsons L H, Shulteis G, Hyytia P, Lorang M T, Bloom F E, Koob G F (1996) Ethanol self administration restores withdrawal associated deficiencies in accumbal dopamine and 5-hydroxytryptamine release in dependent rats. J Neurosci 16:3474-3485.

Weiss F, Porrino L J (2002) Behavioral neurobiology of alcohol addiction: recent advances and challenges. J Neurosci 22:3332-3337.

Yao L, Arolfo M P, Dohrman D P, Jiang Z, Fan P, Fuchs S, Janak P H, Gordon A S, and Diamond I (2002) βγ dimers mediate synergy of dopamine D₂ and adenosine A₂ receptor-stimulated PKA signaling and regulate ethanol consumption. Cell 109:733-743.

TABLE 1
Effect of DMPX, DPCPX and eticlopride on number of inactive lever presses.
DMPX

| Saline | 1 mg/kg | 3 mg/kg | 5 mg/kg | 7 mg/kg | 10 mg/kg | 20 mg/kg |
|---|---|---|---|---|---|---|
| n = 18 | n = 10 | n = 10 | n = 8 | n = 8 | n = 10 | n = 8 |
| 2.92 ± 0.45 | 2.60 ± 0.85 | 1.50 ± 0.37 | 3.56 ± 1.29 | 3.93 ± 2.17 | 3.60 ± 1.76 | 3.87 ± 0.64 |

DPCPX

| Vehicle | 0.125 mg/kg | 0.25 mg/kg | 0.5 mg/kg |
|---|---|---|---|
| n = 7 | n = 7 | n = 7 | n = 7 |
| 2.71 ± 0.56 | 1.78 ± 0.62 | 2.36 ± 1.08 | 2.86 ± 0.88 |

ETICLOPRIDE

| Saline | 0.005 mg/kg | 0.007 mg/kg | 0.01 mg/kg |
|---|---|---|---|
| n = 5 | n = 5 | n = 5 | n = 5 |
| 2.00 ± 0.67 | 2.00 ± 0.58 | 1.00 ± 0.41 | 1.83 ± 0.98 |

Values are given as mean ± SEM

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of mitigating one or more components of addictive behavior associated with chronic consumption of a substance of abuse, or withdrawal therefrom, by a mammal, said method comprising:
administering to said mammal exhibiting one or more components of addictive behavior an adenosine A2A receptor antagonist in an amount sufficient to ameliorate said one or more components of addictive behavior, where said A2A receptor antagonist is not caffeine.

2. The method of claim 1, wherein said adenosine A2A receptor antagonist is selected from the group consisting of (−)-R,S)-mefloquine, 3,7-Dimethyl-1-propargylxanthine (DMPX), 3-(3-hydroxypropyl)-7-methyl-8-(m-methoxystyryl)-1-propargylxanthine (MX2), 3-(3-hydroxypropyl)-8-(3-methoxystyryl)-7-methyl-1-propargylxanthin phosphate disodium salt (MSX-3), 7-methyl-8-styrylxanthine derivatives, SCH 58261, KW-6002, aminofuryltriazolo-triazinylaminoethylphenol (ZM 241385), and 8-chlorostyrylcaffeine, KF17837, VR2006, istradefylline, VER-1 1135, VER-6409, VER 6440, VER 6489, VER 6623, VER 6947, VER 7130, VER 7146, VER 7448, VER 7835, VER 8177, pyrazolo [4,3-e)1,2,4-triazolo[1,5-c]pyrimidines, and 5-amino-imidazolo-[4,3-e]-1,2,4-triazolo[1,5-c]pyrimidines.

3. The method of claim 1, wherein said antagonist does not substantially antagonize the adenosine A1A receptor.

4. The method of claim 1, wherein said substance of abuse is selected from the group consisting of ethanol, an opiate, a cannabinoid, nicotine, and a stimulant.

5. The method of claim 1, wherein said substance of abuse is selected from the group consisting morphine, heroin, marijuana, hashish, cocaine, and amphetamines.

6. The method of claim 1, wherein said substance of abuse is ethanol.

7. The method of claim 1, wherein said component of addictive behavior is chronic self-administration of said substance of abuse.

8. The method of claim 1, wherein said component of addictive behavior is craving for said substance of abuse.

9. The method of claim 1, wherein said component of addictive behavior is reinstatement of seeking behavior for said substance of abuse.

10. The method of claim 1, wherein said mammal is a mammal engaging in chronic consumption of a substance of abuse.

11. The method of claim 1, wherein said mammal is a mammal that has ceased chronic consumption of a substance of abuse.

12. The method of claim 1, wherein said mammal is a mammal undergoing one or more symptoms of withdrawal.

13. The method of claim 1, wherein said mammal is a human.

14. The method of claim 1, wherein said mammal is a human not suffering from Parkinson's disease.

15. The method of claim 1, wherein said antagonist is administered systemically.

16. The method of claim 1, wherein said antagonist is administered by a route selected from the group consisting of oral administration, nasal administration, rectal administration, intraperitoneal injection, intravascular injection, subcutaneous injection, transcutaneous administration, inhalation administration, and intramuscular injection.

17. The method of claim 1, wherein said antagonist is formulated as a unit dosage formulation.

18. The method of claim 1, wherein said antagonist is formulated as a time-release formulation.

19. The method of claim 1, wherein said method further comprises administering a dopamine D2 receptor antagonist in conjunction with said adenosine A2A receptor antagonist.

20. The method of claim 19, wherein said dopamine D2 receptor antagonist is administered before said adenosine A2A receptor antagonist.

21. The method of claim 19, wherein said dopamine D2 receptor antagonist is administered after said adenosine A2A receptor antagonist.

22. The method of claim 19, wherein said dopamine D2 receptor antagonist is administered simultaneously with said adenosine A2A receptor antagonist.

23. The method of claim 19, wherein said adenosine A2A receptor antagonist and said dopamine D2 receptor antagonist are formulated as a single compound formulation.

24. The method of claim 19, wherein said dopamine D2 receptor antagonist is selected from the group consisting of butaclamol, chlorpromazine, domperidone, fluphenazine, haloperidol, heteroaryl piperidines, metoclopramide, olanzapine, perospirone hydrochloride hydrate, phenothiazine, pimozide, quetiapine, risperidone, sertindole, sulpiride, ziprasidone, and zotepine.

25. A composition for mitigating one or more components of addictive behavior associated with chronic consumption of a substance of abuse, or withdrawal therefrom, by a mammal, said composition comprising:
an adenosine A2A receptor antagonist; and
a dopamine D2 receptor antagonist.

26. The composition of claim 25, wherein said dopamine D2 receptor antagonist is selected from the group consisting of butaclamol, chlorpromazine, domperidone, fluphenazine, haloperidol, heteroaryl piperidines, metoclopramide, olanzapine, perospirone hydrochloride hydrate, phenothiazine, pimozide, quetiapine, risperidone, sertindole, sulpiride, ziprasidone, and zotepine.

27. The composition of claim 25, wherein said adenosine A2A receptor antagonist is selected from the group consisting of (−)-R,S)-mefloquine, 3,7-Dimethyl-1-propargylxanthine (DMPX), 3-(3-hydroxypropyl)-7-methyl-8-(m-methoxystyryl)-1-propargylxanthine (MX2), 3-(3-hydroxypropyl)-8-(3-methoxystyryl)-7-methyl-1-propargylxanthin phosphate disodium salt (MSX-3), 7-methyl-8-styrylxanthine derivatives, SCH 58261, KW-6002, aminofuryltriazolo-triazinylaminoethylphenol (ZM 241385), and 8-chlorostyrylcaffeine, KF17837, VR2006, istradefylline, VER-11135, VER-6409, VER 6440, VER 6489, VER 6623, VER 6947, VER 7130, VER 7146, VER 7448, VER 7835, VER 8177, pyrazolo [4,3-e]1,2,4-triazolo[1,5-c]pyrimidines, and 5-amino-imidazolo-[4,3-e]-1,2,4-triazolo[1,5-c]pyrimidines.

28. A kit for mitigating one or more components of addictive behavior associated with chronic consumption of a substance of abuse, or withdrawal therefrom, by a mammal, said kit comprising:
a container containing one or more adenosine A2A receptor antagonists wherein at least one of said one or more adenosine A2A receptor antagonists is not caffeine; and
instructional materials teaching the use of said adenosine A2A receptor antagonists in the treatment of substance abuse in a mammal.

29. The kit of claim 28, wherein said one or more adenosine A2A receptor antagonists comprises an antagonist selected from the group consisting of (−)-R,S)-mefloquine, 3,7-Dimethyl-1-propargylxanthine (DMPX), 3-(3-hydroxypropyl)-7-methyl-8-(m-methoxystyryl)-1-propargylxanthine (MX2), 3-(3-hydroxypropyl)-8-(3-methoxystyryl)-7-methyl-1-propargylxanthin phosphate disodium salt (MSX-3), 7-methyl-8-styrylxanthine derivatives, SCH 58261, KW-6002, aminofuryltriazolo-triazinylaminoethylphenol (ZM 241385), and 8-chlorostyrylcaffeine, KF17837, VR2006, istradefylline, VER-11135, VER-6409, VER 6440, VER 6489, VER 6623, VER 6947, VER 7130, VER 7146, VER 7448, VER 7835, VER 8177, pyrazolo [4,3-e]1,2,4-triazolo[1,5-c]pyrimidines, and 5-amino-imidazolo-[4,3-e]-1,2,4-triazolo[1,5-c]pyrimidines.

30. The kit of claim 28, wherein said antagonist does not substantially antagonize the adenosine A1A receptor.

31. The kit of claim 28, wherein said substance of abuse is selected from the group consisting of ethanol, an opiate, a cannabinoid, nicotine, and a stimulant.

32. The kit of claim 28, wherein said substance of abuse is selected from the group consisting of morphine, heroin, marijuana, hashish, cocaine, and amphetamines.

33. The kit of claim 28, wherein said substance of abuse is ethanol.

34. The kit of claim 28, wherein said component of addictive behavior is chronic self-administration of said substance of abuse.

35. The kit of claim 28, wherein said component of addictive behavior is craving for said substance of abuse.

36. The kit of claim 28, wherein said component of addictive behavior is reinstatement of seeking behavior for said substance of abuse.

37. The kit of claim 28, wherein said antagonist is formulated for administration by a route selected from the group consisting of oral administration, nasal administration, rectal administration, intraperitoneal injection, intravascular injection, subcutaneous injection, transcutaneous administration, inhalation administration, and intramuscular injection.

38. The kit of claim 28, wherein said antagonist is formulated as a unit dosage formulation.

39. The kit of claim 28, wherein said antagonist is formulated as a time-release formulation.

40. A method of screening for an agent that inhibits one or more components of addictive behavior associated with chronic consumption of a substance of abuse said method comprising:
providing one or more test agents; and
screening said test agents for the ability to inhibit adenosine A2A receptor expression or activity wherein inhibition of adenosine A2A receptor expression or activity indicates that said one or more test agents are candidate agents for inhibiting one or more components of addictive behavior associated with chronic consumption of a substance of abuse, or withdrawal therefrom.

41. The method of claim 5, wherein said screening comprises screening said test agent for the ability to bind to an A2A receptor.

42. The method of claim 41, wherein said screening comprises further screening said test agent for the ability to inhibit operant self-administration of said substance of abuse.

43. The method of claim 41, wherein said screening comprises further screening said test agent for the ability to inhibit reinstatement of seeking behavior for said substance of abuse.

44. The method of any one of claims 40-43, wherein said substance of abuse is selected from the group consisting of ethanol, an opiate, a cannabinoid, nicotine, and a stimulant.

45. The method of any one of claims 40-43, wherein said substance of abuse is selected from the group consisting of morphine, heroin, marijuana, hashish, cocaine, and amphetamines.

46. A method of screening for an agent that inhibits one or more components of addictive behavior associated with chronic consumption of a substance of abuse said method comprising:
providing one or more putative adenosine A2A receptor antagonists; and
screening said test agents for the ability to inhibit one or more components of an addictive behavior associated with chronic consumption of a substance of abuse or withdrawal therefrom.

47. The method of claim 46, wherein said screening comprises screening said test agent for the ability to inhibit operant self-administration of said substance of abuse.

48. The method of claim 46, wherein said screening comprises further screening said test agent for the ability to inhibit reinstatement of seeking behavior for said substance of abuse.

49. The method of any one of claims 46-48, wherein said substance of abuse is selected from the group consisting of a stimulant, an opiate, a cannabinoid, nicotine, and ethanol.

50. The method of any one of claims 46-48, wherein said substance of abuse is selected from the group consisting of morphine, heroin, marijuana, hashish, cocaine, and amphetamines.
